(19) United States
(12) Patent Application Publication
(10) Pub. No.: US 2026/0331264 A1
(43) Pub. Date: Oct. 22, 2026

(54) Methods and Systems for Machine Learning-Based Image Transformation

(71) Applicant: AVAILORTHO LLC, Holden, MA (US)

(72) Inventors: Christopher J. Vinton, Holden, MA (US); Qazi Rushdy Ahmad, Sharon, MA (US); Prashanth Balassoubramaniane, North Grafton, MA (US)

(21) Appl. No.: 19/651,059

(22) Filed: Apr. 17, 2026

Related U.S. Application Data
(60) Provisional application No. 63/791,204, filed on Apr. 18, 2025.

(51) Int. Cl. G16H 30/40; G06N 3/0464; G06T 3/14; G06T 7/10; G06T 7/33

(52) U.S. Cl. CPC G16H 30/40; G06N 3/0464; G06T 3/14; G06T 7/10; G06T 7/33; G06T 2207/10116; G06T 2207/20084; G06T 2207/30008

(57) ABSTRACT

Embodiments perform machine learning (ML)-based image transformation. An embodiment receives an operative hip image and a non-operative hip image The operative hip image and the non-operative hip image are associated with a patient. The method then, via an ML model, segments the received non-operative hip image and the received operative hip image to identify a set of features. Based on the set of features, at least one transformation parameter is determined. In turn, based on the at least one transformation parameter determined, the method transforms the received operative hip image and the received non-operative hip image into an aligned overlay image.

### RELATED APPLICATION

This application claims the benefit of U.S. Provisional Application No. 63/791,204, filed on Apr. 18, 2025. The entire teachings of the above application are incorporated herein by reference.

### BACKGROUND

Hip replacement is one of the most common orthopedic operations, in which the hip joint is replaced by a prosthetic implant. Generally, a hip replacement is conducted to relieve arthritis or avascular necrosis pain or for some types of hip fractures. Hip replacement surgery can be a total replacement (total hip arthroplasty (THA)) or a hemi/semi (half) replacement (hemiarthroplasty). THA replaces both the acetabulum and the femoral head, while hemiarthroplasty only replaces the femoral head. Patient satisfaction with hip replacement surgery is very high overall but is dependent on accurate restoration of anatomy.

### SUMMARY

Example embodiments relate to an autonomous machine learning (ML)/artificial intelligence (AI) system for real-time X-ray analysis and surgical guidance in orthopedics.

Example embodiments provide several advantages over existing approaches such as, for example, the VELYS® system (DePuy Synthes, Inc., Raynham, MA) or the OrthoGrid® system (OrthoGrid Systems, Inc., Salt Lake City, UT).

Example embodiments provide unique advancements over existing approaches. For instance, a traditional system primarily offers static grid overlays for manual alignment reference. In contrast, example embodiments employ real-time, autonomous landmark recognition and dynamic realignment of anatomical structures. Such a capability significantly enhances surgical precision by continuously updating an imported surgical X-ray and realigning it back to its neutral reference state. The process of example embodiments also compensates dynamically for changes in intraoperative patient positioning and C-arm movement.

Additionally, compared to a conventional system, which relies heavily on user input for alignment protocols, example embodiments deliver ML/AI image recognition and segmentation, thus providing faster and more reliable image alignment. Example embodiments also provide independent rotational segmentation of a femoral component. This feature allows for precise, independent adjustment and verification of femoral positioning intraoperatively by rotating a femoral segment around a true anatomic center of rotation at the hip joint. Such a feature also enables a system of embodiments to correct an X-ray image to a true, neutral, anatomic state for more accurate calculation of anatomic differences during a surgical procedure. This is a significant advantage over the existing system, which only allows rotation of an entire image around an arbitrary point, and over another traditional system, which does not allow for rotational correction.

Example embodiments uniquely integrate landmark-based calculations to derive precise translation and rotation metrics, thus offering a significantly improved level of accuracy and reproducibility. This detailed, real-time feedback and dynamic correction capability sets example embodiments apart from existing technologies, for instance by addressing known shortcomings in manual adjustments and rigid, user driven positioning inherent in traditional systems.

An example embodiment is directed to a computer-implemented method for ML-based image transformation. The method begins by receiving an operative hip image and a non-operative hip image; optionally, the method further receives a pelvis, e.g., a neutral pelvis, image. The operative hip image and the non-operative hip image are associated with a patient. The optional pelvis image is associated with the patient. The method then, via an ML model, segments the received non-operative hip image and the received operative hip image, and optionally the received pelvis image, to identify a set of features. Based on the set of features, at least one transformation parameter is determined. In turn, based on the at least one transformation parameter determined, the method transforms the received operative hip image and the received non-operative hip image into an aligned overlay image.

In an example embodiment, transforming the received operative hip image and the received non-operative hip image may include inverting the received non-operative hip image. According to another example embodiment, transforming the received operative hip image and the received non-operative hip image may include overlaying the received operative hip image and the received non-operative hip image. In an embodiment, the overlay image may be based on a pelvic portion of the patient anatomy.

According to an example embodiment, at least one transformation parameter determined may include a horizontal translation parameter and a vertical translation parameter. Transforming the received operative hip image and the received non-operative hip image may include, based on the horizontal translation parameter and the vertical translation parameter, aligning the received operative hip image with the received non-operative hip image. In one such embodiment, the method may further include receiving a pelvis image. The pelvis image may be associated with the patient. The method may further include, via the ML model, segmenting the received pelvis image to identify a pubis feature. The method may further include, based on the pubis feature, determining a pubic orientation parameter. The method may further include, based on a predefined orientation parameter and the determined pubic orientation parameter, determining an intersection parameter. The method may further include, based on the pubis feature and the determined intersection parameter, determining the horizontal translation parameter and the vertical translation parameter. According to another such embodiment, the method may further include receiving a translation adjustment value from a user and, based on the received translation adjustment value, updating at least one of the determined horizontal translation parameter and vertical translation parameter.

In an example embodiment, the set of features may include at least one non-operative femoral feature and at least one operative femoral feature. The at least one transformation parameter determined may include a rotation parameter. Transforming the received operative hip image and the received non-operative hip image may include, based on a rotation center parameter and the rotation parameter, aligning the at least one operative femoral feature with the at least one non-operative femoral feature. According to one such embodiment, the at least one operative femoral feature includes at least one isolated femoral anatomic segment and at least one isolated femoral implant segment. Aligning the at least one operative femoral feature with the at least one non-operative femoral feature may include exclusively applying the rotation parameter to the at least one isolated femoral anatomic segment and at least one isolated femoral implant segment. In another such embodiment, the set of features further may include an acetabular cup feature. The method may further include, based on the acetabular cup feature, determining the rotation center parameter. According to yet another such embodiment, the method may further include receiving an acetabular cup size value and a distance value. The method may further include, based on the determined rotation center parameter, the received acetabular cup size value, and the received distance value, determining a scaled rotation center parameter. The method may further include updating the rotation center parameter based on the determined scaled rotation center parameter. In one such embodiment, determining the least one transformation parameter may include, based on a non-operative femoral axis value and an operative femoral axis value, determining the rotation parameter. The rotation parameter may be configured to align the non-operative femoral long axis parallel to the operative femoral long axis. According to another such embodiment, the method may further include receiving a rotation adjustment value from a user and, based on the received rotation adjustment value, updating the determined rotation parameter. In yet another such embodiment, the set of features may include a non-operative greater trochanter feature, a non-operative femur feature, an operative greater trochanter feature, and an operative femur feature. The method may further include, based on the non-operative greater trochanter feature and the non-operative femur feature, determining the non-operative femoral axis value. The method may further include, based on the operative greater trochanter feature and the operative femur feature, determining the operative femoral axis value.

According to an example embodiment, the set of features may include a non-operative upper greater trochanter feature, a non-operative lower lesser trochanter feature, an operative upper greater trochanter feature, and an operative lower lesser trochanter feature. The method may further include, based on a horizontal orientation parameter and (i) the non-operative upper greater trochanter feature and the operative upper greater trochanter feature or (ii) the non-operative lower lesser trochanter feature and the operative lower lesser trochanter feature, determining a leg length difference value. In one such embodiment, the method may further include receiving a pelvis image. The pelvis image may be associated with the patient. The method may further include, via the ML model, segmenting the received pelvis image to identify a teardrop feature and a pubis feature. The method may further include, based on the teardrop feature, determining the horizontal orientation parameter. The method may further include, based on the determined horizontal orientation parameter and the pubis feature, determining a vertical orientation parameter. According to another such embodiment, the method may further include, based on a non-operative femoral axis value, the non-operative upper greater trochanter feature, and the non-operative lower lesser trochanter feature, determining the horizontal orientation parameter.

In an example embodiment, the ML model may include at least one convolutional neural network (CNN). The at least one CNN may preferably include a YOLOv8 model.

According to an example embodiment, the method may further include outputting the aligned overlay image on a display.

In an example embodiment, receiving the operative hip image and the non-operative hip image may include obtaining hip X-ray data associated with the patient and processing the obtained hip X-ray data to generate the operative hip image and the non-operative hip image. According to one such embodiment, the obtained hip X-ray data may be interoperative data of the patient acquired from a C-arm based X-ray device. The method may further include displaying the obtained hip X-ray data on a user interface in real-time.

According to an example embodiment, receiving the operative hip image and the non-operative hip image may further include: (1) obtaining operative hip X-ray data associated with the patient; (2) processing the obtained operative hip X-ray data to generate the operative hip image; (3) obtaining non-operative hip X-ray data associated with the patient; and (4) processing the obtained non-operative hip X-ray data to generate the non-operative hip image. In one such embodiment, the obtained operative hip X-ray data and obtained non-operative hip X-ray data may be interoperative data of the patient acquired from a C-arm based X-ray device. The method may further include displaying the obtained operative hip X-ray data and obtained non-operative hip X-ray data on a user interface in real-time.

Another example embodiment is directed to a computer-based system for ML-based image transformation. The system includes a processor and a memory with computer code instructions stored or held thereon. The processor and the memory, with the computer code instructions, are configured to cause the system to implement any embodiments, or combination of embodiments, described herein.

Yet another example embodiment is directed to a computer program product for ML-based image transformation. The computer program product includes a non-transitory computer-readable medium with computer code instructions stored thereon. The computer code instructions are configured, when executed by a processor, to cause an apparatus associated with the processor to implement any embodiments, or combination of embodiments, described herein. As understood by one skilled in the art, one or more processors may execute the computer code instructions to cause the apparatus to implement an embodiment.

It is noted that embodiments of the method, system, and computer program product may be configured to implement any embodiments, or combination of embodiments, described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is an example system architecture according to an embodiment.

FIG. 2 is a flow diagram of an example process of capturing live images according to an example embodiment

FIG. 3 is a flow diagram of an example process of image upload and processing for anteroposterior (AP) pelvis images according to an example embodiment.

FIG. 4A is an example AP pelvis image.

FIG. 4B illustrates example segmentation of the image of FIG. 4A according to an embodiment.

FIG. 5 is a flow diagram of an example process of horizontal and vertical line population on an AP pelvis image according to an embodiment.

FIG. 6A is another example AP pelvis image.

FIG. 6B illustrates example processing of the image of FIG. 6A according to an embodiment.

FIG. 7 is a flow diagram of an example process of vertical line drawing along a long axis of a femur according to an embodiment.

FIG. 8A is an example AP non-operative hip image.

FIG. 8B illustrates example processing of the image of FIG. 8A according to an embodiment.

FIG. 9 is a flow diagram of an example process of determining a scaling factor according to an embodiment.

FIG. 10A is an example AP operative hip image.

FIG. 10B illustrates example processing of the image of FIG. 10A according to an embodiment.

FIG. 11 is a flow diagram of an example process of calculating a center of rotation according to an embodiment.

FIG. 12A is another example AP operative hip image.

FIG. 12B illustrates example processing of the image of FIG. 12A according to an embodiment.

FIG. 13 is a flow diagram of an example process of aligning operative and non-operative AP hip images according to an embodiment.

FIGS. 14A and 14B illustrate an example vertical femoral line and operative femoral long axis prior to and after alignment, respectively, according to an embodiment.

FIG. 15 is a flow diagram of an example process of femoral rotation adjustment according to an embodiment.

FIG. 16 is a flow diagram of an example process of determining an alternative horizontal line according to an embodiment.

FIG. 17 is a flow diagram of an example process of calculating leg length discrepancy according to an embodiment.

FIG. 18 is a flow diagram of an example process of femoral offset calculation according to an embodiment.

FIG. 19 is a flowchart of a method for machine learning-based image transformation according to an example embodiment.

FIG. 20 is a schematic view of a computer network in which embodiments may be implemented.

FIG. 21 is a block diagram illustrating an example embodiment of a computer node in the computer network of FIG. 20.

### DETAILED DESCRIPTION

A description of example embodiments follows.

As used herein, the term “operative” may refer to an object or body part of, relating to, involving, or resulting from a medical operation, e.g., surgery.

As used herein, the term “non-operative” may refer to an object or body part that is not of, relating to, involving, or resulting from a medical operation, e.g., surgery. For example, in a total hip arthroplasty procedure, the operative hip is the hip on that side of the body on which the surgical procedure is or will be performed, while the non-operative hip is the hip of the other side of the body.

As used herein, the term “neutral pelvis” may refer to a neutral pelvic rotation as determined by one or more of the below non-limiting example criteria:
a) The coccyx in line with the symphysis pubis.
b) Symmetry of the obturator foramen.
c) Symmetry of the teardrop.
d) Horizontal alignment of the trans teardrop line.

### INTRODUCTION

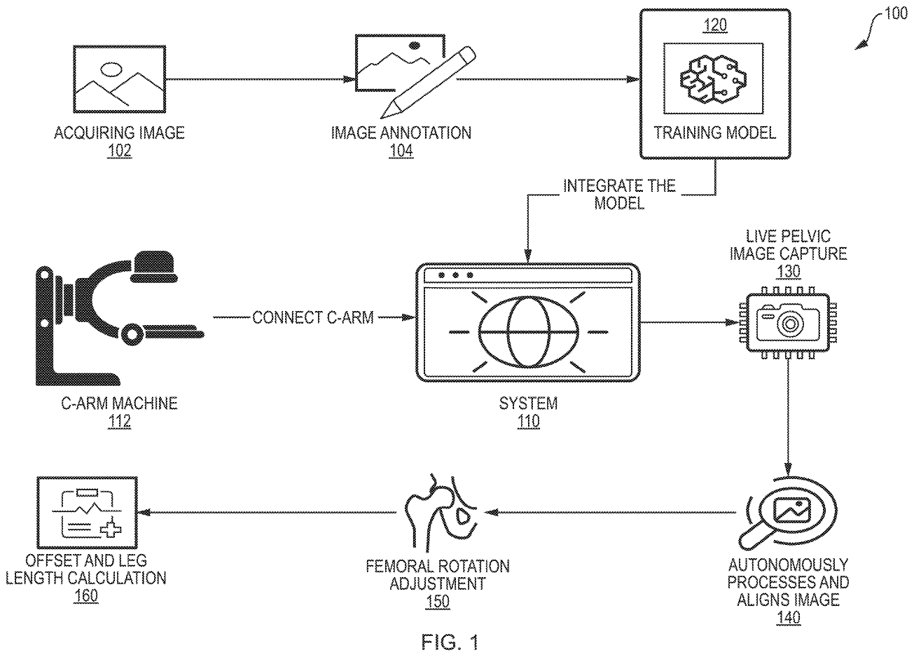

FIG. 1 is an example system architecture 100 according to an embodiment. As shown in FIG. 1, the architecture 100 includes a computer-based system 110. In an embodiment, the system 110 may be implemented at least in part via the Django® web framework and/or the JavaScript® language; other known frameworks and languages are also suitable. To continue, the system 110 may be integrated with a machine learning (ML)/artificial intelligence (AI) model 120 that is trained with annotated images 104 generated from acquired images 102. The system 110 may be connected to a C-arm X-ray machine 112. The system 110 may be configured to capture 130 live pelvic image(s). The system 110 may be further configured to perform autonomous processing and/or alignment 140 of the captured 130 image(s). The system 110 may be further configured to perform femoral rotation adjustment 150 of the processed/aligned 140 image(s). The system 110 may be further configured to perform offset and/or leg length calculation(s) 160 based on the adjusted 150 image(s).

In an example embodiment, an ML/AI-powered system (e.g., 110) may be designed to autonomously process (e.g., 140) and analyze anteroposterior (AP) pelvis and AP hip X-rays, thereby enhancing accuracy and efficiency in total hip arthroplasty procedures, for non-limiting example. The system may integrate directly with a C-arm X-ray machine (e.g., 112) to capture live pelvic images (e.g., 130) and provide real-time analysis to ensure precise limb alignment. The system may automatically detect and segment baseline pelvic, non-operative hip, and operative hip X-rays. The system may establish these three X-rays (i.e., pelvic, operative hip, and non-operative hip) in a coordinate system and may automatically overlap, align (e.g., 140), and/or fit operative and/or non-operative images.

An example embodiment may be built on an ML/AI model (e.g., 120) that is trained with annotated X-ray datasets (e.g., 104) within, e.g., the Amazon Web Services® (AWS) framework. Other known frameworks are also suitable. To continue, one such embodiment may leverage advanced computer vision algorithms to process (e.g., 140), align (e.g., 140), and/or analyze medical images with high accuracy. In an embodiment, once trained, an example ML/AI model (e.g., 120) may operate in real time to deliver automated, consistent assessments to support clinical decision-making.

By combining ML/AI-driven image analysis with real-time surgical guidance, an example embodiment can enhance precision, reduce variability in implant placement, and improve patient outcomes in orthopedic procedures by more accurately reestablishing patient anatomy.

Provided hereinbelow is a description of example features of embodiments and their implementations.

### Example Autonomous Recognition and Segmentation of AP Pelvis X-Rays

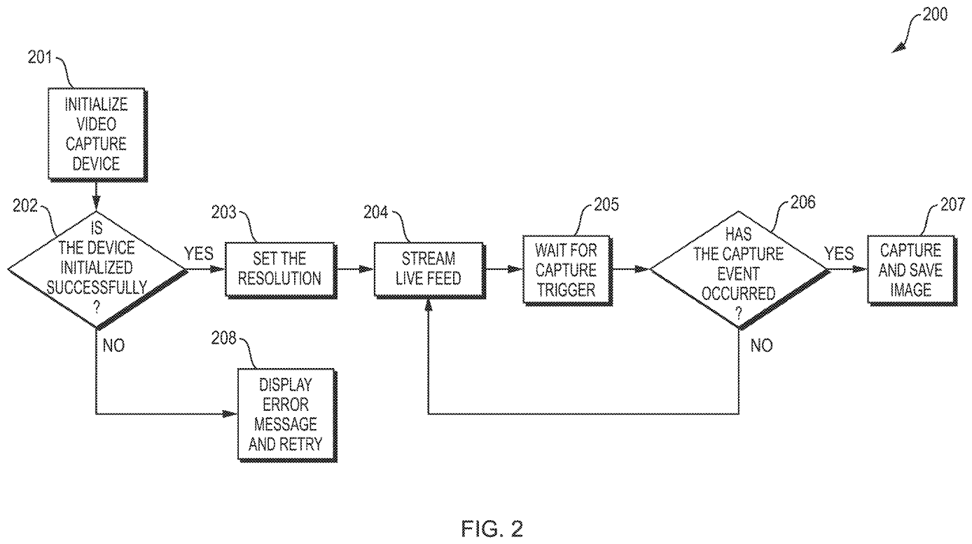

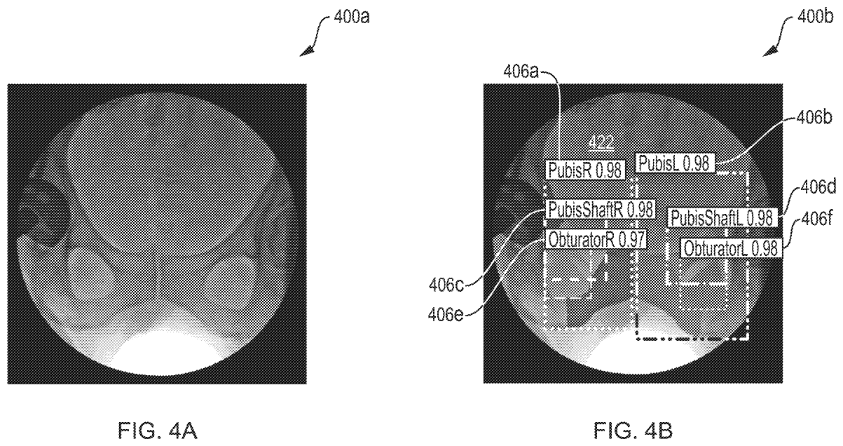

FIG. 2 is a flow diagram of an example process 200 of capturing live images (e.g., AP pelvis X-ray image 400a (FIG. 4A)) according to an embodiment. In an embodiment, the process 200 may be implemented by the system 110 (FIG. 1), such as in the pelvic/hip image capture module 130 (FIG. 1).

The process 200 begins at step 201 by initializing a video capture device. In an embodiment, step 201 may include performing one or more of the below non-limiting example operations:
a) Establish a connection with the device, e.g., C-arm imaging device 112 (FIG. 1); other known video capture and imaging devices are also suitable.
b) Configure the capture device index and/or resolution settings.
c) Start the image streaming process.

At step 202, if the process 200 determines that the device is not successfully initialized, an error message is displayed at step 208 and the process 200 returns to step 201 to reattempt device initialization. In an embodiment, step 202 may include displaying an output such as a blank screen or color bars if an appropriate feed is not received from the capture device; other known display types are also suitable. If device initialization and video capture are successful, then an image may be displayed that mirrors or replicates an output screen of the capture device.

Otherwise, if the device is successfully initialized at step 202, the process 200 continues to step 203 and configures resolution settings of the device. In an embodiment, a setting of 980×980 resolution may be used; other known resolution settings are also suitable. According to another embodiment, output from the device may be captured through a BNC connection and fed to a DVI input; other known connection types are also suitable.

At step 204, the process 200 receives streaming data of a live feed from the device. In an embodiment, step 204 may include (1) fetching or retrieving, e.g., continuously fetching or retrieving, frames from the device feed and/or (2) displaying a live preview of the retrieved frames on a user interface. According to another embodiment, step 204 may displaying a live image matching the last image obtained on the capture device (e.g., C-arm 112 (FIG. 1)) or any image that is placed on the C-arm monitor from the C-arm memory. In one such embodiment, the image may be a greyscale image of known resolution and may represent an X-ray taken in surgery.

The process 200 then waits for a capture trigger at step 205. At step 206, if the process 200 determines that a capture event has not occurred, the process 200 returns to step 204 and receives further streaming data.

Otherwise, if a capture event is determined to have occurred at step 206, e.g., via user trigger of a capture event, the process 200 continues to step 207 and captures and saves live image(s) (e.g., 400a (FIG. 4A)) from the streaming 204 data. In an embodiment, step 207 may include performing either or both of the below non-limiting example operations:
a) Storing the current frame from the device feed as an image, e.g., a high-resolution image. In an embodiment, the image may be stored in a database within program working memory. According to one such embodiment, the database may be specific to the patient for which the program is being used. Other known storage types and locations are also suitable.
b) Saving the stored image in a predetermined location, e.g., a predefined filesystem directory with an identifiable filename, such as a filename that includes or is generated based at least in part on a patient identifier (ID) or patient case ID. According to an embodiment, the location (e.g., filesystem path) of the captured 207 image may be saved in a database, such as in a database table with row or entries indexed by patient identifiers IDs or patient case IDs.

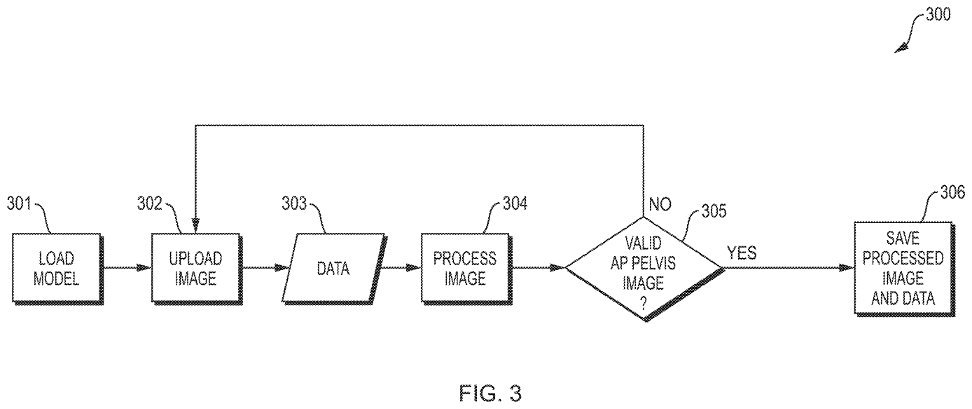

FIG. 3 is a flow diagram of an example process 300 of image upload and processing for AP pelvis images according to an embodiment. In an embodiment, the process 300 may be implemented by the system 110 (FIG. 1), such as in the processing/alignment module 140 (FIG. 1).

The process 300 begins at step 301 by loading an ML/AI model, e.g., 120 (FIG. 1). In an embodiment, the model may include a version 8 You Only Look Once (YOLOv8TM) convolutional neural network (CNN) model for real-time image segmentation. YOLOv8's efficient single-pass architecture processes images in real-time, while leveraging anchor boxes and multi-scale feature maps to detect landmarks at varying scales, thereby ensuring robustness across X-ray images with different angles and resolutions. Other known image segmentation models are also suitable. According to another embodiment, the model may be trained on annotated datasets with labeled anatomical landmarks, such as the teardrop, obturator, pubis, ischium, femur, acetabulum, acetabular cup, lesser trochanter, and greater trochanter, for non-limiting examples, and may be configured to detect these segments with high accuracy.

Next, at step 302, an image, e.g., an image captured/saved using the process 200 (FIG. 2), is uploaded to the loaded 301 model.

At step 303, data (e.g., pixel values for the image in the x/y plane) is obtained or extracted from the uploaded 302 image.

In turn, at step 304, the uploaded 302 image is processed based on the obtained 303 data. According to an embodiment, step 304 may include performing image segmentation and/or recognition with the loaded 301 model to output bounding box coordinates and/or confidence scores for detected landmark(s) (e.g., landmarks 406a-406f (FIG. 4B)), along with a mask polygon that encapsulates the anatomical structure itself.

At step 305, if the processed 304 image is determined not to be a valid AP pelvis image, the process 300 returns to step 302 and uploads another image. In an embodiment, step 305 may include segmenting the processed 304 image and/or performing image recognition via the loaded 301 model to determine if the image is or includes a pelvis. If the model does not recognize the image at step 305, an operator may be prompted to upload a valid image and/or an error message may be displayed. Otherwise, if the processed 304 image is determined to be a valid AP pelvis image at step 305, the process 300 continues to step 306 and saves the processed 304 image/data. In an embodiment, step 306 may include storing the processed 304 image/data in a database within program working memory. According to one such embodiment, the database may be specific to the patient for which the program is being used. Other known storage types and locations are also suitable.

FIG. 4A is an example AP pelvis X-ray image 400a.

FIG. 4B is an image 400b illustrating example segmentation of anatomical landmarks 406a-406f in the image 400a according to an embodiment. As shown in FIG. 4B, the segmented landmarks may include right pubis 406a, left pubis 406b, right pubis shaft 406c, left pubis shaft 406d, right obturator 406e, and left obturator 406f, for non-limiting examples. In an embodiment, a segmented landmark may have an associated confidence score, such as confidence score 422 associated with the segmented right pubis 406a.

An example embodiment may identify and segment AP pelvis X-ray images (e.g., 400a) and recognize distinct anatomical landmarks (e.g., 406a-406j) in the segmented images (e.g., 400b), such as via the processes 200 and/or 300 (FIGS. 2 & 3). In an embodiment, an ML/AI model (e.g., 120 (FIG. 1)) may be utilized to orient the pelvis in an X/Y planar system.

Provided below is example pseudocode in a Python®-like language for autonomous recognition and segmentation of AP pelvis images, which may be used as part of implementing the process 300; other known programming languages are also suitable.
Input: ap_pelvis_image: the X-ray image into the model for prediction
Output: result_list: bounding boxes, confidence scores, labels for detected landmarks and processed X-ray image with visual markers
Data: model: Load YOLOv8 Model with trained weights
Function: segment_and_recognize(ap_pelvis_image):
   model=initialize the YOLOv8 model instance
   results=model.predict(ap_pelvis_image) #Extract bounding boxes, confidence scores, labels and save the segmented image for retrieval
   return results

### Example Recognition of Distinct Landmarks and Orientation of AP Hip X-Rays

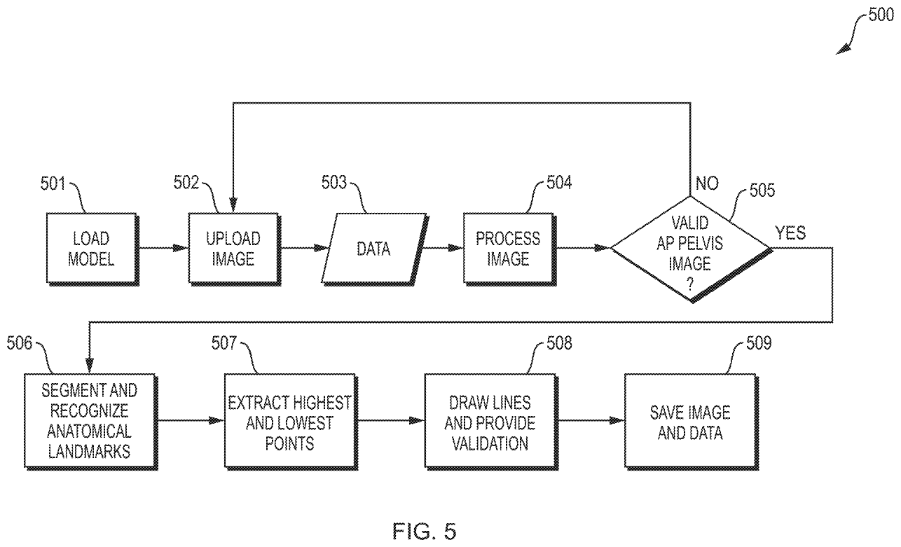

FIG. 5 is a flow diagram of an example process 500 of horizontal and vertical line population on an AP pelvis image according to an embodiment. In an embodiment, the process 500 may be implemented by the system 110 (FIG. 1), such as in the processing/alignment module 140 (FIG. 1).

The process 500 begins at step 501 by loading an ML/AI model, e.g., 120 (FIG. 1). In an embodiment, the model may include a YOLOv8 CNN model for real-time image segmentation. YOLOv8's efficient single-pass architecture processes images in real-time, while leveraging anchor boxes and multi-scale feature maps to detect landmarks at varying scales, thereby ensuring robustness across X-ray images with different angles and resolutions. Other known image segmentation models are also suitable. According to another embodiment, the model may be trained on annotated datasets with labeled anatomical landmarks, such as the teardrop, obturator, pubis, ischium, femur, acetabulum, acetabular cup, lesser trochanter, and greater trochanter, for non-limiting examples, and may be configured to detect these segments with high accuracy.

At step 502, an image, e.g., an image captured/saved using the process 200 (FIG. 2), is uploaded to the loaded 501 model.

Next, at step 503, data (e.g., pixel values for the image in the x/y plane) is obtained or extracted from the uploaded 502 image.

At step 504, the uploaded 502 image is then processed based on the obtained 503 data. According to an embodiment, step 504 may include performing image segmentation and/or recognition with the loaded 501 model to output bounding box coordinates and/or confidence scores for detected landmark(s), e.g., each detected landmark, along with a mask polygon that encapsulates the anatomical structure itself.

If the processed 504 image is determined not to be a valid AP pelvis image at step 505, the process 500 returns to step 502 and uploads another image. According to an embodiment, step 505 may include segmenting the processed 504 image and/or performing image recognition via the loaded 501 model to determine if the image is or includes a pelvis. If the model does not recognize the image at step 505, an operator may be prompted to upload a valid image and/or an error message may be displayed. Otherwise, if the processed 504 image is determined to be a valid AP pelvis image at step 505, the process 500 continues to step 506.

At step 506, the process 500 segments and/or recognizes anatomical landmark(s) in the processed 504 image. In an embodiment, step 506 may include using the loaded 501 model to carry out pubis segmentation by identifying the pubis region.

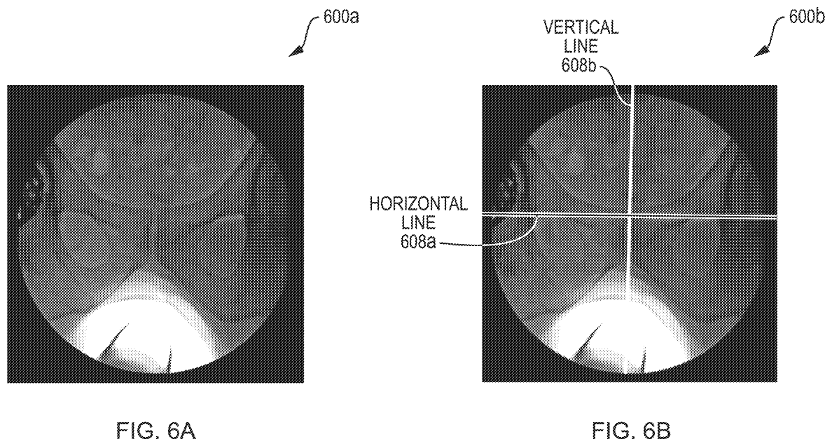

In turn, at step 507, the process 500 extracts highest and/or lowest point(s) from the segmented 506 image. At step 508, the process 500 draws line(s) (e.g., 608a & 608b (FIG. 6B)) in and/or provides validation of the processed 506 image. In an embodiment, validating the processed 506 image at step 508 may include displaying the image and/or prompting an operator to accept the image as validated. According to an embodiment, steps 507 and/or 508 may include calculating an angle between the X-axis and the pubic line and/or performing an X/Y orientation that uses the distinct recognized 506 landmark(s) and calculated angle to orient a subsequently acquired AP hip X-ray.

The process 500 then concludes at step 509 by saving the validated 508 image and the obtained 503 data. In an embodiment, step 509 may include storing the validated 508 image in a database within program working memory. According to one such embodiment, the database may be specific to the patient for which the program is being used. Other known storage types and locations are also suitable.

FIG. 6A is another example AP pelvis X-ray image 600a.

FIG. 6B is an image 600b illustrating example horizontal 608a and vertical 608b line population for procedural assistance in the image 600a according to an embodiment.

An example embodiment may autonomously recognize a distinct landmark on an AP pelvis X-ray that will also be identifiable on a subsequently acquired AP hip X-ray, such as via the process 500 (FIG. 5). For instance, an embodiment may segment a portion of the pubis and create a polygonal shape that defines a reproducible best fit straight line. An intersecting angle may be measured between a previously defined x-axis and the newly defined pubic line. Using the distinct landmark common to the AP pelvis and AP hip, the segmented pubic polygon, and the best fit line, as well as the newly defined intersecting angle, an embodiment may orient the AP hip X-ray in an X/Y planar system to match the orientation of the original AP pelvis.

An example embodiment may use Python, optionally in conjunction with one or more Python libraries such as NumPy®, for mathematical calculations. Other known programming languages and libraries are also suitable. Non-limiting examples of calculations may include determining desired point(s) from a segmented landmark (e.g., 507 (FIG. 5)), drawing a horizontal line (e.g., 508 (FIG. 5)), and determining a slope of the line to then establish an orthogonal vertical line (e.g., 508 (FIG. 5)).

Provided below is example pseudocode in a Python-like language for drawing horizontal and/or vertical lines in AP pelvis images, which may be used as part of implementing the process 500; other known programming languages are also suitable.

```
Input: ap_pelvis_image : the X-ray image to be processed for
line drawing
Output: processed_ap_pelvis_image _image : Image populated with
the horizontal and vertical lines
Data: model : Load YOLOv8 Model with trained weights
Function: draw_horizontal_and_vertical_line (ap_pelvis_image) :
  model = initialize the YOLOv8 model instance
  results = model.predict (ap_pelvis_image) # Extract bounding
boxes, confidence scores, labels, and save the segmented image
for retrieval
  all_boxes = A numpy list initialization to convert tensors
to numpy data type
  for each result in results do
    sorted_indices = Sort the class labels
    boxes_np = Extract the bounding box data from tensors
    masks_np = Extract the polygon data from tensors
    all_boxes = Stack the bounding box data from result
sorted_mask_arrays = Sort the polygon list based on the class
indices found
sorted_box_arrays = Sort the bounding box list based on the
class indices found
lowest_points = Extract all the lowest points from the polygon
list sorted_mask_arrays
teardrop_left_x, teardrop_left_y = Assign the lowest x and y
data for the left teardrop by index
teardrop_right_x, teardrop_right_y = Assign the lowest x and y
data for the right teardrop by index
image = Open the ap_pelvis_image to draw on
# Draw the horizontal line based on assigned points
image.line ( (teardrop_left_x, teardrop_left_y) ,
(teardrop_right_x, teardrop_right_y) )
slope_original = Calculate slope of the horizontal line
slope_perpendicular = Calculate the negative inverse of the
slope_original
pubis_left = Extract the leftmost x value of the left pubis
pubis_right = Extract the rightmost x value of the right pubis
start_x_vertical = Calculate the middle x value using pubis_left
and pubis_right
start_y_vertical = Take the lowest y value from the bounding box
data of the pubis
end_x_vertical = Influence start_x_vertical with
slope_perpendicular
end_y_vertical = Define a ending height based on the image that
is being processed
# Draw the vertical line orthogonal to the horizontal line based
on assigned points
image.line ( (start_x_vertical, start_y_vertical) ,
(end_x_vertical, end_y_vertical) )
return image
```

### Example Identification of Femoral Long Axis

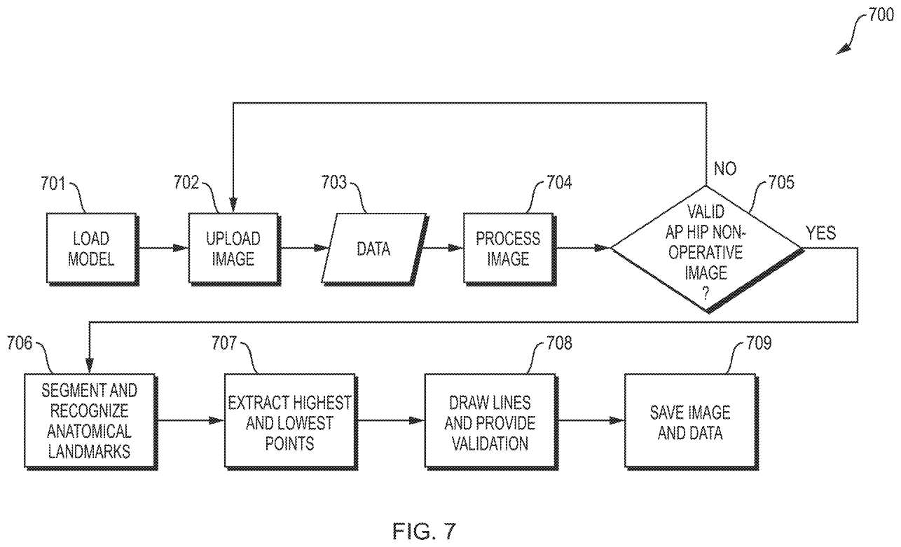

FIG. 7 is a flow diagram of an example process 700 of vertical line drawing along a long axis of a femur according to an embodiment. In an embodiment, the process 700 may be implemented by the system 110 (FIG. 1), such as in processing/alignment module 140 (FIG. 1).

The process 700 begins at step 701 by loading an ML/AI model, e.g., 120 (FIG. 1). In an embodiment, the model may include a YOLOv8 CNN model for real-time image segmentation. YOLOv8's efficient single-pass architecture processes images in real-time, while leveraging anchor boxes and multi-scale feature maps to detect landmarks at varying scales, thereby ensuring robustness across X-ray images with different angles and resolutions. Other known image segmentation models are also suitable. According to another embodiment, the model may be trained on annotated datasets with labeled anatomical landmarks, such as the teardrop, obturator, pubis, ischium, femur, acetabulum, acetabular cup, lesser trochanter, and greater trochanter, for non-limiting examples, and may be configured to detect these segments with high accuracy.

At step 702, an image, e.g., an image captured/saved using the process 200 (FIG. 2), is uploaded to the loaded 701 model.

Next, at step 703, data (e.g., pixel values for the image in the x/y plane) is obtained or extracted from the uploaded 702 image.

At step 704, the uploaded 702 image is then processed based on the obtained 703 data. According to an embodiment, step 704 may include performing image segmentation and/or recognition with the loaded 701 model to output bounding box coordinates and/or confidence scores for detected landmark(s), e.g., each detected landmark, along with a mask polygon that encapsulates the anatomical structure itself.

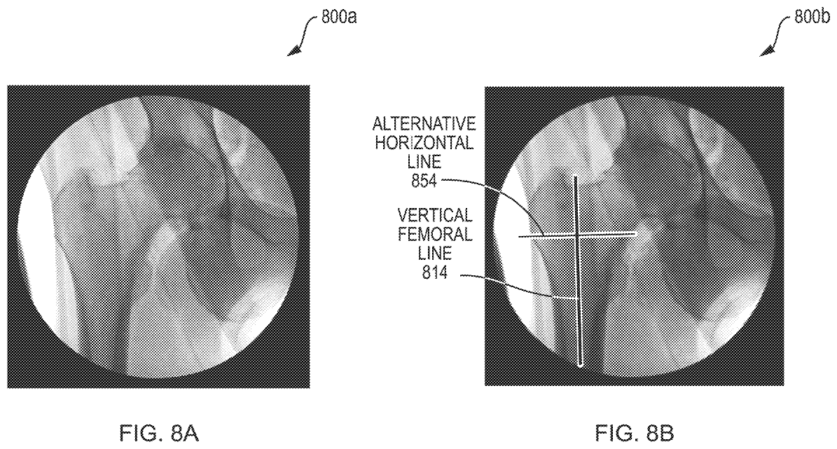

If the processed 704 image is determined not to be a valid AP hip non-operative image at step 705, the process 700 returns to step 702 and uploads another image. According to an embodiment, step 705 may include segmenting the processed 704 image and/or performing image recognition via the loaded 701 model to determine if the image is or includes a hip. If the model does not recognize the image at step 705, an operator may be prompted to upload a valid image and/or an error message may be displayed. Otherwise, if the processed 704 image is determined to be a valid AP hip non-operative image (e.g., 800a (FIG. 8)) at step 705, the process 700 continues to step 706.

At step 706, the process 700 segments and/or recognizes anatomical landmark(s) in the processed 704 image. In an embodiment, step 706 may include using the loaded 701 model to identify femoral landmarks using edge detection.

In turn, at step 707, the process 700 extracts highest and/or lowest point(s) from the segmented 706 image. At step 708, the process 700 draws line(s) in and/or provides validation of the processed 706 image. In an embodiment, validating the processed 706 image at step 708 may include displaying a visual line for an operator to, e.g., review, validate, accept, and proceed. According to an embodiment, steps 707 and/or 708 may include determining the desired points, such as via NumPy calculations, for non-limiting example, and/or drawing a vertical line (e.g., 814 (FIG. 8)) along the long axis of the femur.

The process 700 then concludes at step 709 by saving the validated 708 image and the obtained 703 data. In an embodiment, step 709 may include storing the validated 708 image in a database within program working memory. According to one such embodiment, the database may be specific to the patient for which the program is being used. Other known storage types and locations are also suitable.

FIG. 8A is an example AP hip X-ray image 800a.

FIG. 8B is an example AP hip image 800b illustrating alternative horizontal line 854 and vertical femoral line 814 population for procedural assistance for the image 800a according to an embodiment.

An example embodiment may autonomously identify and mark long axes (e.g., 814) of femurs on operative AP hip and/or non-operative AP hip X-rays (e.g., 800a) for later angular correction and/or limb alignment calculations, such as via the process 700 (FIG. 7).

Provided below is example pseudocode in a Python-like language for drawing femoral vertical lines in AP hip non-operative images, which may be used as part of implementing the process 700; other known programming languages are also suitable.

```
Input: non_operative_image : the X-ray image to be processed for
line drawing
Output: processed_non_operative_image : Image populated with
the vertical line along the long axis of the femur
Data: model : Load YOLOv8 Model with trained weights
Function :
draw_femoral_vertical_line (non_operative_image) :
model = initialize the YOLOv8 model instance
results = model.predict (non_operative_image) # Extract bounding
boxes, confidence scores, labels
and save the segmented image for retrieval
all_boxes = A numpy list initialization to convert tensors numpy
data type
for each result in results do
  sorted_indices = Sort the class labels
  boxes_np = Extract the bounding box data from tensors
  masks_np = Extract the polygon data from tensors
  all_boxes = Stack the bounding box data from result
sorted_mask_arrays = Sort the polygon list based on the class
indices found
sorted_box_arrays = Sort the bounding box list based on the
class indices found
lowest_points = Extract all the lowest points from the polygon
list sorted_mask_arrays
greater_trochanter_x, greater_trochanter_y = Extract the point
for the greater trochanter
femur_bottom_x, femur_bottom_y = Extract the point for the femur
image = Open the non operative image to draw on
# Draw the vertical line based on assigned points along the long
axis of the femur
image.line ( (greater_trochanter_x, greater_trochanter_y) ,
(femur_bottom_x, femur_bottom_y) )
return image
```

### Example Determination of Best Fit Circle Around Acetabular Cup

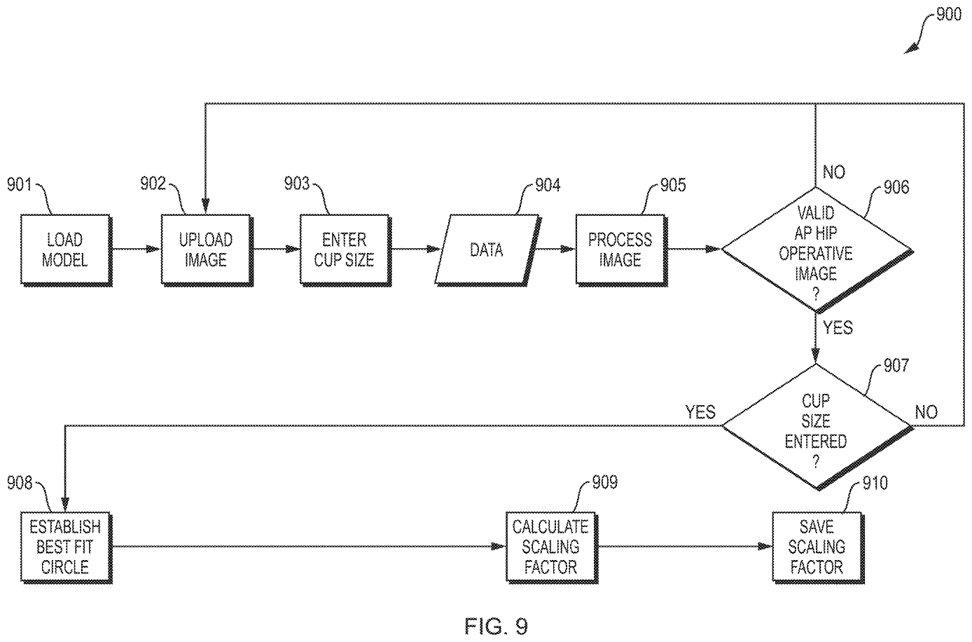

FIG. 9 is a flow diagram of an example process 900 of determining a scaling factor according to an embodiment. In an embodiment, the process 900 may be implemented by the system 110 (FIG. 1), such as in processing/alignment module 140 (FIG. 1).

The process 900 begins at step 901 by loading an ML/AI model, e.g., 120 (FIG. 1). In an embodiment, the model may include a YOLOv8 CNN model for real-time image segmentation. YOLOv8's efficient single-pass architecture processes images in real-time, while leveraging anchor boxes and multi-scale feature maps to detect landmarks at varying scales, thereby ensuring robustness across X-ray images with different angles and resolutions. Other known image segmentation models are also suitable. According to another embodiment, the model may be trained on annotated datasets with labeled anatomical landmarks, such as the teardrop, obturator, pubis, ischium, femur, acetabulum, acetabular cup, lesser trochanter, and greater trochanter, for non-limiting examples, and may be configured to detect these segments with high accuracy.

At step 902, an image, e.g., an image captured/saved using the process 200 (FIG. 2), is uploaded to the loaded 901 model.

Next, at step 903, cup size data is received.

At step 904, data (e.g., pixel values for the image in the x/y plane) is obtained or extracted from the uploaded 902 image.

The uploaded 902 image is then processed at step 905 based on the obtained 904 data. According to an embodiment, step 905 may include performing image segmentation and/or recognition with the loaded 901 model to output bounding box coordinates and/or confidence scores for detected landmark(s), e.g., each detected landmark, along with a mask polygon that encapsulates the anatomical structure itself.

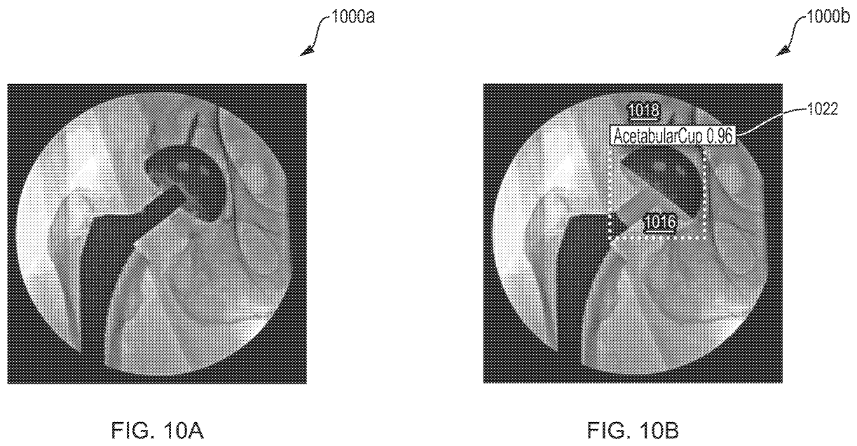

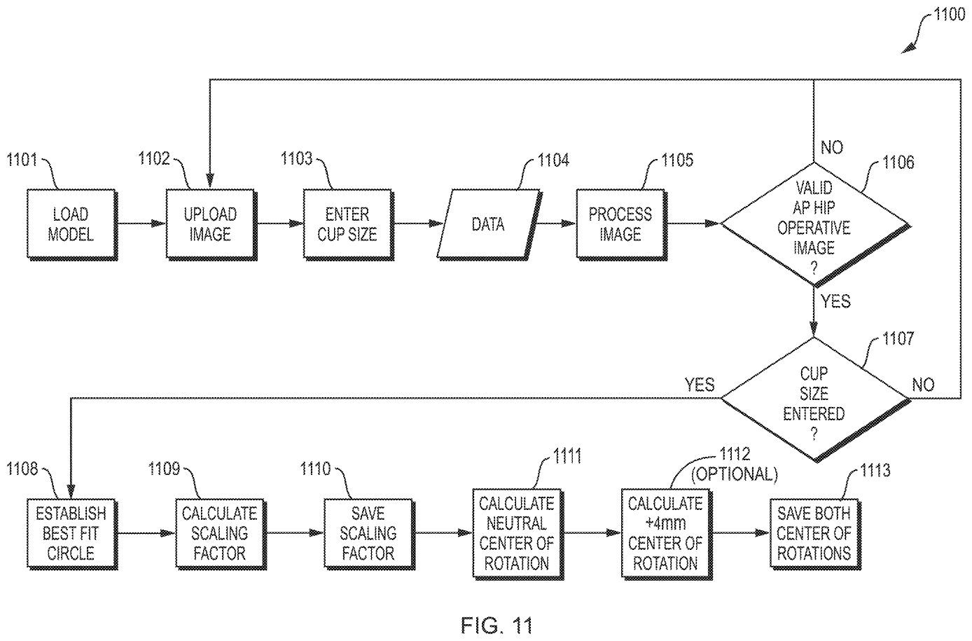

If the processed 905 image is determined not to be a valid AP hip operative image at step 906, the process 900 returns to step 902 and uploads another image. According to an embodiment, step 906 may include segmenting the processed 905 image and/or performing image recognition via the loaded 901 model to determine if the image is or includes a hip. If the model does not recognize the image at step 906, an operator may be prompted to upload a valid image and/or an error message may be displayed. Otherwise, if the processed 905 image is determined to be a valid AP hip operative image (e.g., 1000a (FIG. 10A)) at step 906, the process 900 continues to step 907.

If it is determined that the received 903 cup size data does not indicate user input to enter the acetabular cup diameter at step 907, the process 900 returns to step 902 and uploads another image. According to an embodiment, step 907 may include determining whether the user input has been received. Otherwise, if the received 903 cup size data indicates user input or entry of a diameter of the acetabular cup at step 907, the process 900 continues to step 908.

At step 908, the process 900 establishes a best fit circle (e.g., 1016 (FIG. 10B)) around the acetabular cup (e.g., 1018 (FIG. 10B)) based on the processed 905 image. In an embodiment, step 908 may include using the loaded 901 model to segment or achieve a best fit circle based on the model's training data.

In turn, at step 909, the process 900 calculates a scaling factor. According to an embodiment, step 909 may include calculating the ratio of (i) the diameter (e.g., in pixels) of the established 908 best fit circle and (ii) the received 903 cup size data. In an embodiment, the received 903 cup size data may include the cup diameter in millimeters, which may be used to scale pixels to millimeters for subsequent length calculations.

The process 900 then concludes at step 910 by saving the calculated 909 scaling factor. In an embodiment, step 910 may include saving the calculated 909 scaling factor in working memory of a program or application. Other known storage locations are also suitable. FIG. 10A is an example AP hip operative X-ray image 1000a.

FIG. 10B is an example image 1000b illustrating a best fit circle 1016 for an acetabular cup 1018 in the image 1000a according to an embodiment. As shown in FIG. 10B, the segmented acetabular cup 1018 may have an associated confidence score 1022.

An example embodiment may establish a true reference scale for later limb alignment calculations by autonomously placing a best fit circle (e.g., 1016) around an acetabular cup (e.g., 1018) of known diameter provided as input by a user, such as via the process 900 (FIG. 9).

Provided below is example pseudocode in a Python-like language for determining best fit circles around acetabular cups in AP hip operative images, which may be used as part of implementing the process 900; other known programming languages are also suitable.

```
Input: operative_image : the X-ray image to be processed for
line drawing
Output: processed_operative_image : bounding boxes, confidence
scores, labels for best fit acetabular circle and processed
X-ray image with visual markers
Data: model : Load YOLOv8 Model with trained weights
Function: best_fit_circle (operative_image) :
  model = initialize the YOLOv8 model instance
  results = model.predict (operative_image) # Extract bounding
boxes, confidence scores, labels and save the segmented image
for retrieval
  cup_size_input = user input in millimeters
  cup_size_detected = the diameter of the cup as per the
model in pixels
  scaling_factor = the cup diameter in pixels divided by the
cup size in mm
return scaling_factor
```

### Example Center of Rotation Determination and Non-Central Liner Adjustment

FIG. 11 is a flow diagram of an example process 1100 of calculating a center of rotation according to an embodiment. In an embodiment, the process 1100 may be implemented by the system 110 (FIG. 1), such as in processing/alignment module 140 (FIG. 1).

The process 1100 begins at step 1101 by loading an ML/AI model, e.g., 120 (FIG. 1). In an embodiment, the model may include a YOLOv8 CNN model for real-time image segmentation. YOLOv8's efficient single-pass architecture processes images in real-time, while leveraging anchor boxes and multi-scale feature maps to detect landmarks at varying scales, thereby ensuring robustness across X-ray images with different angles and resolutions. Other known image segmentation models are also suitable. According to another embodiment, the model may be trained on annotated datasets with labeled anatomical landmarks, such as the teardrop, obturator, pubis, ischium, femur, acetabulum, acetabular cup, lesser trochanter, and greater trochanter, for non-limiting examples, and may be configured to detect these segments with high accuracy.

At step 1102, an image, e.g., an image captured/saved using the process 200 (FIG. 2), is uploaded to the loaded 1101 model.

Next, at step 1103, cup size data is received.

At step 1104, data (e.g., pixel values for the image in the x/y plane) is obtained or extracted from the uploaded 1102 image.

The uploaded 1102 image is then processed at step 1105 based on the obtained 1104 data. According to an embodiment, step 1105 may include performing image segmentation and/or recognition with the loaded 1101 model to output bounding box coordinates and/or confidence scores for detected landmark(s), e.g., each detected landmark, along with a mask polygon that encapsulates the anatomical structure itself.

If the processed 1105 image is determined not to be a valid AP hip operative image at step 1106, the process 1100 returns to step 1102 and uploads another image. According to an embodiment, step 1106 may include segmenting the processed 1105 image and/or performing image recognition via the loaded 1101 model to determine if the image is or includes a hip. If the model does not recognize the image at step 1106, an operator may be prompted to upload a valid image and/or an error message may be displayed. Otherwise, if the processed 1105 image is determined to be a valid AP hip operative image at step 1106, the process 1100 continues to step 1107.

If it is determined that the received 1103 cup size data does not indicate user input to enter the acetabular cup diameter at step 1107, the process 1100 returns to step 1102 and uploads another image. According to an embodiment, step 1107 may include determining whether the user input has been received. Otherwise, if the received 1103 cup size data indicates user input or entry of a diameter of the acetabular cup at step 1107, the process 1100 continues to step 1108.

At step 1108, the process 1100 establishes a best fit circle around the acetabular cup based on the processed 1105 image. In an embodiment, step 1108 may include using the loaded 1101 model to segment or achieve a best fit circle based on the model's training data.

In turn, at step 1109, the process 1100 calculates a scaling factor. According to an embodiment, step 1109 may include calculating the ratio of (i) the diameter (e.g., in pixels) of the established 1108 best fit circle and (ii) the received 1103 cup size data.

At step 1110, the process 1100 saves the calculated 1109 scaling factor. In an embodiment, step 1110 may include storing the calculated 1109 scaling factor in a database within program working memory. According to one such embodiment, the database may be specific to the patient for which the program is being used. Other known storage types and locations are also suitable.

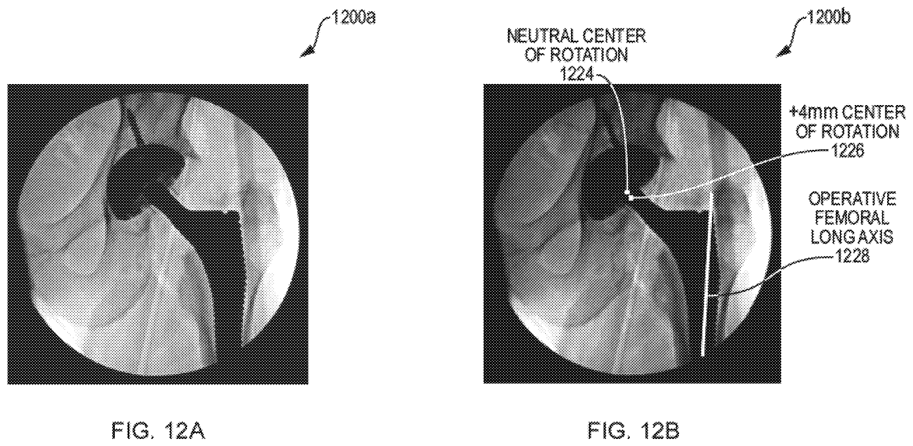

The process 1100 then calculates a center of rotation (e.g., 1224 (FIG. 12B)) at step 1111. In an embodiment, step 1111 may include utilizing Python, optionally in conjunction with one or more Python libraries, to determine the center of rotation from the established 1108 best fit circle.

Optionally, at step 1112, the process 1100 may calculate a +4 mm variation (e.g., 1226 (FIG. 12B)) on the calculated 1111 center of rotation; other known variation sizes are also suitable.

At step 1113, the process 1100 concludes by saving the calculated 1111 center of rotation and any optionally calculated 1112 variation. In an embodiment, step 1113 may include storing the calculated 1111 center of rotation and any optionally calculated 1112 variation in a database within program working memory. According to one such embodiment, the database may be specific to the patient for which the program is being used. Other known storage types and locations are also suitable.

FIG. 12A is an example input AP hip operative X-ray image 1200a.

FIG. 12B is an example image 1200b illustrating a center of rotation (e.g., neutral center of rotation) 1224 determination for the image 1200a according to an embodiment. As shown in FIG. 12B, an example embodiment may optionally determine a +4 mm variant 1226 of the center of rotation 1224; other known variant sizes are also suitable. In an embodiment, an operative femoral long axis 1228 may also be determined.

An example embodiment may autonomously define centers of rotation (e.g., 1224) of acetabular cups and determine edges of cups that deviate from the best fit circles. In an embodiment, a line may be defined or established by the two points on either side of the hemispheric cup where that hemispheric cup exits the best fit circle that was fit around the hemispheric cup. According to one such embodiment, the defined line may represent the flat edge of the hemispheric cup. Using such an initial defined line, a new line may be defined that is 90 degrees to the defined line and intersects a previous center of rotation. An embodiment may autonomously define an optional +4 mm scenario (e.g., 1226) to move a center of rotation when using a non-central liner (also referred to as an offset/asymmetric liner), i.e., an acetabular liner designed with thicker material on one side than the other, to shift the center of rotation of the femoral head away from the center of the cup. Scenarios with other known sizes are also suitable.

Provided below is example pseudocode in a Python-like language for determining centers of rotation in AP hip operative images, which may be used as part of implementing the process 1100; other known programming languages are also suitable.

```
Input: operative_image : the X-ray image to be processed for
line drawing
Output: processed_operative_image : bounding boxes, confidence
scores, labels for best fit acetabular circle, center of
rotation, center of rotation +4mm and processed X-ray image with
visual markers
Data: model : Load YOLOv8 Model with trained weights
Function: center_of_rotation (operative_image) :
  model = initialize the YOLOv8 model instance
  results = model.predict (operative_image) # Extract bounding
boxes, confidence scores, labels and save the segmented image
for retrieval
  cup_size_input = user input in millimeters
  cup_size_detected = the diameter of the cup as per the
model in pixels
  scaling_factor = the cup diameter in pixels divided by the
cup size in mm
  center_of_rotation_x = the central x value of the bounding
box of the cup
  center_of_rotation_y = the central y value of the bounding
box of the cup
  distance = 4 * scaling factor # Since we want to move 4mm
  center_of_rotation_x_four_mm = calculate it by multiplying
the distance with the cosine of the angle of line orthogonal to
the diameter of the circle
  center_of_rotation_y_four_mm = calculate it by multiplying
the distance with the sine of the angle of line orthogonal to
the diameter of the circle
  return center_of_rotation_x, center_of_rotation_y,
center_of_rotation_x_four_mm, center_of_rotation_y_four_mm
```

### Example Cup Inclination Determination and Anteversion Calculation

An example embodiment may autonomously recognize ellipses on the edges of acetabular cups. Using value D (the long axis of an ellipse) and a previously defined X/Y planar coordinate system, another example embodiment may determine or report cup inclination in reference to the true horizontal plane of a patient. Using an ellipse, an example embodiment may recognize D, as well as d (the short axis of an ellipse), and compute cup anteversion A using the following example equation: A=arcsin d/D.

In an embodiment, detecting ellipse diameters for anteversion calculations may include using a YOLOv8 image segmentation model to detect an ellipse of an acetabular cup, and using Python libraries such as NumPy to extract d and D, the short and long ellipse diameters, respectively, to calculate anteversion A of the cup. Other known image segmentation models and numerical libraries are also suitable.

### Example Cup Inclination Angle Determination

An example embodiment may autonomously leverage previously identified points on an acetabular cup edge (such as described hereinabove with respect to determining center of rotation) to define a line representing the cup edge. In an embodiment, the cup edge line may be compared to a reference horizontal line to determine or report cup inclination angle.

According to an embodiment, points on an acetabular cup edge may be identified by detecting edge points of the acetabular cup using ML/AI-based image segmentation and extracting two or more points along the cup's edge that define an inclination line.

In another embodiment, a reference horizontal line may be determined or defined using a previously established horizontal reference line (such as at step 508 of the process 500 (FIG. 5)).

According to an embodiment, an acetabular cup inclination angle may be calculated by using known techniques to compute an angle θ between an inclination line and a previously established horizontal reference line.

In an embodiment, a calculated acetabular cup inclination angle may be stored in memory of a system (e.g., 110 (FIG. 1)) and/or a result of the calculation may be displayed for a surgeon's reference as a value in degrees.

Provided below is example pseudocode for determining acetabular cup inclination angles according to an embodiment.
// Step 1: Identify key anatomical landmarks on the acetabular cup
Identify edge point 1 on the acetabular cup
Identify edge point 2 on the acetabular cup
// Step 2: Retrieve the reference horizontal line reference_line=GetReferenceLine( ) // e.g., step 508 of the process 500 (FIG. 5)
// Step 3: Compute the slope of the inclination line
delta_Y=edge point_2_Y−edge point_1_Y
delta_X=edge point_2_X−edge point_1_X
IF delta_X!=0 THEN
   inclination_slope=delta_Y/delta_X
ELSE
   inclination_slope=INFINITY//Handle vertical line case
// Step 4: Calculate the inclination angle
inclination angle rad=a tan(inclination_slope)
inclination angle deg=ConvertToDegrees(inclination angle rad)
// Step 5: Store the calculated cup inclination angle StoreCupInclinationAngle (inclination angle deg)
// Step 6: Display or utilize the inclination angle in further calculations
DisplayCupInclinationAngle(inclination_angle_deg)

### Example Image Alignment of Operative and Non-Operative AP Hip X-Rays

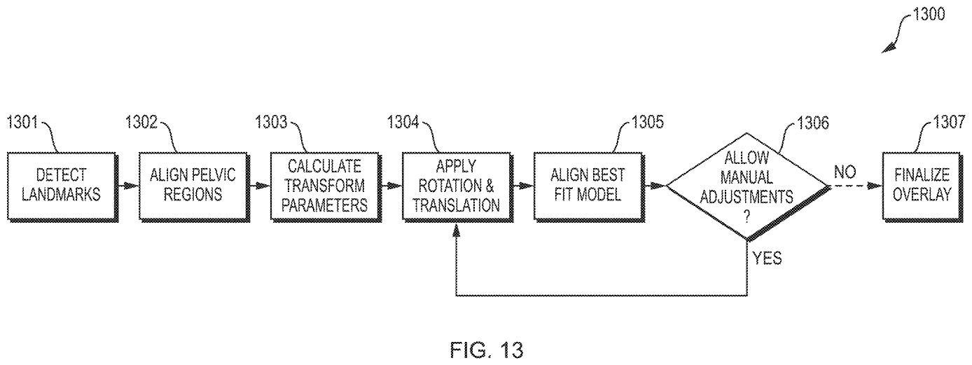

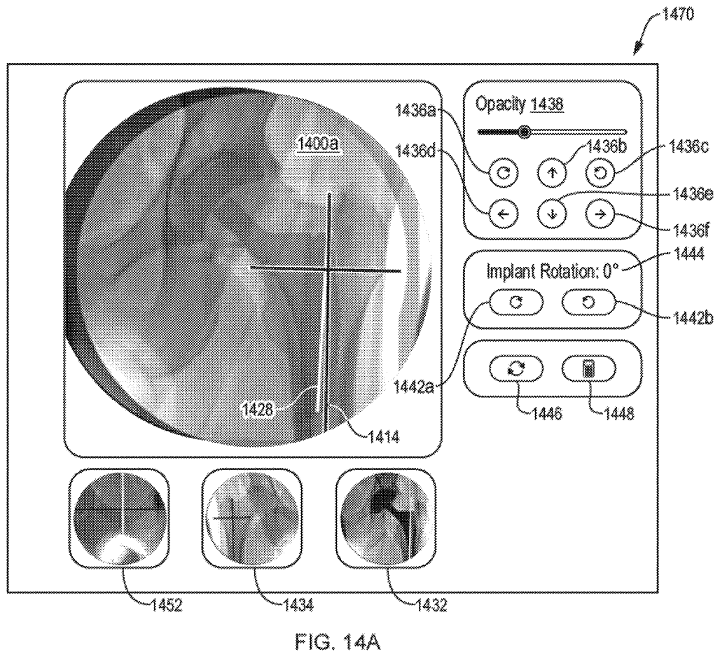

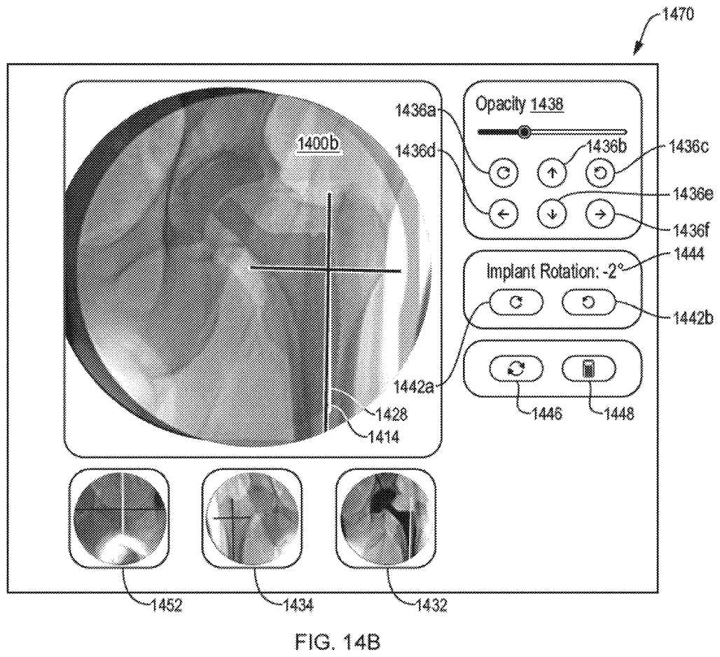

FIG. 13 is a flow diagram of an example process 1300 of aligning operative and non-operative AP hip X-ray images according to an embodiment. In an embodiment, the process 1300 may be implemented by the system 110 (FIG. 1), such as in processing/alignment module 140 (FIG. 1).

The process 1300 begins at step 1301 by detecting landmark(s). At step 1302, the process 1300 aligns pelvic region(s). In an embodiment, steps 1301 and/or 1302 may include detecting landmark(s), e.g., teardrops, obturator foramen, acetabulum, etc., and/or segmenting the pelvic portions in the images.

Next, at step 1303, the process 1300 calculates transform parameter(s). According to an embodiment, step 1303 may include deriving the transformation parameter(s) from calculations based on the detected 1301 landmark(s).

At step 1304, the process 1300 applies rotation(s) and/or translation(s). In an embodiment, step 1304 may include establishing an X/Y planar coordinate system and/or aligning the images by (1) rotating and/or translating the images based on the detected 1301 anatomical features and/or (2) ensuring a consistent scale and/or orientation for proper anatomical overlay 1307.

In turn, at step 1305, the process 1300 aligns a best fit model. According to an embodiment, step 1305 may include aligning the segmented 1301 pelvic anatomy using the calculated 1303 transform parameter(s).

At step 1306, if the process 1300 determines that manual adjustments are allowed, the process 1300 receives one or more manual adjustments (e.g., fine adjustments) input by a user to modify, e.g., rotation/translation 1304 and/or opacity control, etc., for optimizing the overlay 1307, after which the process 1300 returns to step 1304. In an embodiment, the system may be configured such that manual adjustments are determined to be allowed by default at step 1306. According to an embodiment, receiving the manual adjustments at step 1306 may include receiving guidance and/or direction regarding fine adjustments and/or corrections, such as via user manipulation of directional arrows (e.g., 1436b and 1436d-1436f (FIGS. 14A/14B)). Otherwise, if the process 1300 determines that manual adjustments are not allowed at step 1306, the process 1300 continues to step 1307.

The process 1300 concludes at step 1307 by finalizing an overlay. According to an embodiment, step 1307 may include inverting the non-operative AP hip X-ray to facilitate comparison with the operative side.

FIG. 14A depicts an example overlay image 1400a including a vertical femoral line 1414 and an operative femoral long axis 1428 prior to applying an alignment technique, e.g., via the process 1300, according to an embodiment. As shown in FIG. 14A, implant rotation 1444 is 0° and there is inadequate alignment between the vertical femoral line 1414 and operative femoral long axis 1428 in the overlay 1400a. In an embodiment, the image 1400a may be a composite of AP operative hip image 1432 overlaid on AP non-operative hip image 1434. According to another embodiment, the overlay 1400a may be displayed in a user interface (UI) 1470, which may provide interface elements 1436a-1436f (e.g., directional arrow buttons) to control a position and/or rotation and interface element 1438 (e.g., a slider) to control opacity of the non-operative hip image 1434 in the overlay 1400a. Similarly, interface elements 1442a and 1442b (e.g., buttons) may be used to control the implant rotation 1444 in the overlay 1400a. The UI 1470 may further provide interface elements 1446 and 1448 (e.g., buttons) to undo a previous operation and to perform leg length calculations, respectively. In an embodiment, a user may activate or select the calculator button 1448 to signal to a system (e.g., 110 (FIG. 1)) that the user is satisfied with the pelvis overlap and alignment as well as the femoral rotation. According to another embodiment, once the user selects the button 1448, the system may perform leg length calculations based on greater and lesser trochanter and offset calculations based on a long axis of the femur, and display and/or report the results on a summary page for user review and acceptance. An AP pelvis image 1452 may also be displayed in the UI 1470.

FIG. 14B depicts an example overlay image 1400b including the vertical femoral line 1414 and operative femoral long axis 1428 after applying the alignment technique according to an embodiment. As shown in FIG. 14B, the implant rotation 1444 is −2° and there is full alignment between the vertical femoral line 1414 and operative femoral long axis 1428 in the overlay 1400b.

An example embodiment may autonomously invert a non-operative AP hip X-ray and overlap it with an operative AP hip X-ray. In an embodiment, an alignment process may use a best fit model that matches segmented anatomical region(s) of pelvic portion(s) (e.g., of only the pelvic portion(s)) in the X-rays. This may ensure an accurate overlay of the pelvic structures while maintaining anatomical consistency. According to an embodiment, a system (e.g., 110 (FIG. 1)) may allow users to make any manual adjustments (e.g., final manual adjustments) to a completed alignment to fine-tune the positioning as needed.

Provided below is example pseudocode in a Python-like language for determining alignment and/or orientation and generating a best fit model according to an embodiment, which may be used as part of implementing the process 1300, such as at steps 1304 and/or 1305; other known programming languages are also suitable.

```
# Alignment and Orientation
from .models import Overlay
def align_xray (case_id) :
  overlay = Overlay.objects.get (case=case_id)
  # Fetch alignment parameters from stored calculations
  alignment_params = {
    “translation_x”: overlay.translation_x,
    “translation_y”: overlay.translation_y,
    “rotation_angle”: overlay.rotation_angle,
  }
return alignment_params
# Best Fit Model for Overlay
def overlay_xrays (operative_xray, non_operative_xray) :
  inverted_non_oper_xray = invert_xray (non_operative_xray)
  # Apply best fit transformation using stored alignment
parameters
  alignment_params = align_xray (operative_xray.case_id)
  aligned_overlay = apply_transformation (operative_xray,
      inverted_non_oper_xray, alignment_params)
  return aligned_overlay
```

### Example Femoral Rotation Adjustment

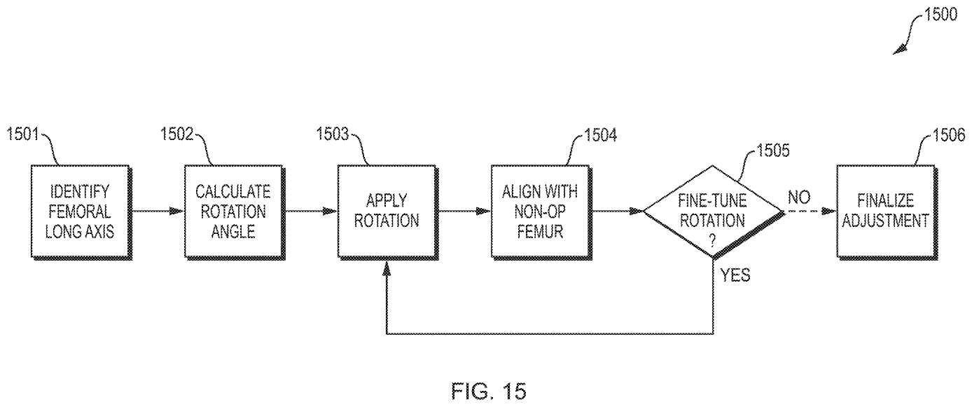

FIG. 15 is a flow diagram of an example process 1500 of femoral rotation adjustment according to an embodiment. In an embodiment, the process 1500 may be implemented by the system 110 (FIG. 1), such as in the adjustment module 150 (FIG. 1).

The process 1500 begins at step 1501 by identifying a non-operative femoral long axis. According to an embodiment, step 1501 may include identifying femoral landmark(s) and/or calculating the long axis.

At step 1502, the process 1500 calculates a rotation angle.

Next, at step 1503, the process 1500 applies the calculated 1502 rotation.

At step 1504, the process 1500 aligns an operative femur with the non-operative femur. In an embodiment, step 1504 may include using a predefined center of rotation (e.g., a neutral center of rotation) or an optional +4 mm variation to rotate the operative femoral segment to achieve parallel alignment with the non-operative femur.

At step 1505, if the process 1500 determines that fine-tuning of the calculated 1502 rotation is allowed, the process 1500 receives one or more manual adjustments or refinements of the rotation from a user to ensure clinical accuracy, after which the process 1500 returns to step 1503. In an embodiment, the system may be configured such that user fine-tuning is determined to be allowed by default at step 1505. Otherwise, if the process 1500 determines that fine-tuning of the calculated 1502 rotation is not allowed at step 1505, the process 1500 continues to step 1506.

The process 1500 concludes at step 1506 by finalizing an adjustment. According to an embodiment, step 1506 may include finalizing the alignment 1504.

An example embodiment may autonomously cut segmented portion(s) of an operative AP hip X-ray containing femoral anatomic segment(s) and a femoral implant. Using a previously defined center of rotation (e.g., a neutral center of rotation) or an optional +4 mm variation, an embodiment may autonomously rotate the cut operative femur and implant until an operative femoral long axis matches parallel to a non-operative femoral long axis and allow users to make final fine adjustments to the rotation.

Provided below is example pseudocode in a Python-like language for determining alignment and/or orientation, applying a rotation, extracting and/or processing femoral segments, calculating long axes, and rotating and/or aligning an operative femur according to an embodiment, which may be used as part of implementing the process 1500; other known programming languages are also suitable.

```
# Alignment and Orientation
from .models import FemurAlignment
def extract_femoral_segment (xray) :
  “ “ “
  Segment the femur and implant from the provided X-ray
image.
  ” ” ”
  femur_segment = segment_femur (xray)
  return femur_segment
def calculate_femoral_axis (femur_segment) :
  “ “ “
  Detect femoral landmarks and compute the long axis of the
femur.
  ” ” ”
  landmarks = detect_femoral_landmarks (femur_segment)
  long_axis = compute_long_axis (landmarks)
  return long_axis
def rotate_femoral_segment (operative_segment, target_axis,
center_of_rotation=“neutral”) :
  “ “ “
  Rotate the operative femur segment to align its long axis
with the target axis.
  Center of rotation can be ‘neutral’ or shifted by +4mm.
  ” ” ”
  current_axis = calculate_femoral_axis (operative_segment)
  # Calculate rotation angle needed to align with target
  rotation_angle = compute_angle_between_axes (current_axis,
target_axis)
  # Determine center of rotation
  rotation_center = get_rotation_center (center_of_rotation)
  # Apply rotation
  rotated_segment = apply_rotation (operative_segment,
rotation_angle, rotation_center)
  return rotated_segment
def align_femurs (operative_xray, non_operative_xray,
center_shift=“+0mm”) :
  “ “ “
  Complete femoral alignment workflow between operative and
non-operative sides.
  ” ” ”
  # Extract and process femoral segments
  operative_femur = extract_femoral_segment (operative_xray)
  non_operative_femur =
extract_femoral_segment (non_operative_xray)
  # Calculate long axes
  target_axis = calculate_femoral_axis (non_operative_femur)
  # Rotate operative femur to align with non-operative
  aligned_femur = rotate_femoral_segment (operative_femur,
target_axis, center_of_rotation=center_shift)
  return aligned_femur
```

### Example Alternative Horizontal Reference Line

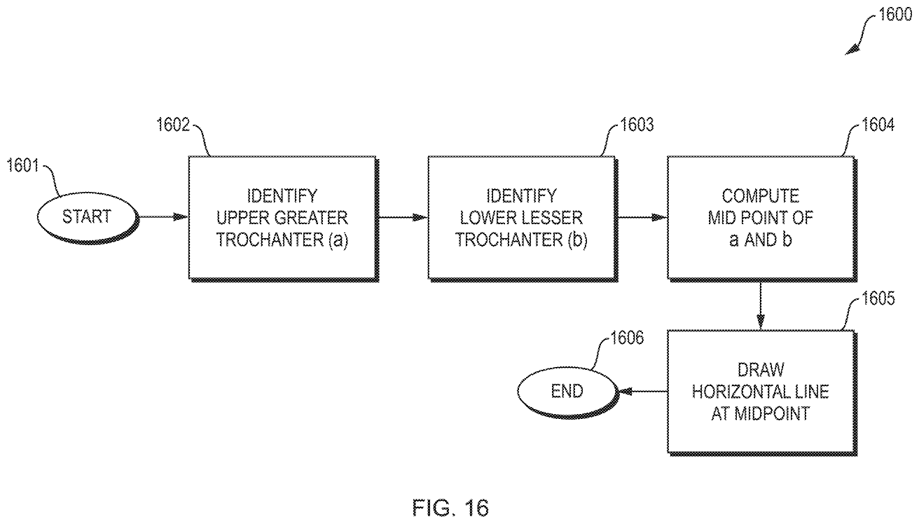

FIG. 16 is a flow diagram of an example process 1600 of determining an alternative horizontal line (e.g., 854 (FIG. 8B)) according to an embodiment. In an embodiment, the process 1600 may be implemented by the system 110 (FIG. 1), such as in the offset/length calculation module 160 (FIG. 1).

The process 1600 starts at step 1601 and identifies an upper greater trochanter (GT) (a) of a non-operative femur at step 1602.

At step 1603, the process 1600 identifies a lower lesser trochanter (LT) (b) of the non-operative femur.

Next, at step 1604, the process 1600 computes a midpoint. According to an embodiment, step 1604 may include defining a midpoint to the Y axis along a long axis of the non-operative femur of (a) and (b).

At step 1605, the process 1600 draws a horizontal line at the computed 1604 midpoint. In an embodiment, if the lower LT (b) was not successfully identified at step 1603, then, at step 1605, the process 1600 may draw a horizontal line 2 cm below from a tip of the upper GT (a) along the Y long axis of the non-operative femur. Additionally or alternately, step 1605 may include drawing a line 90° to the long axis of the non-operative femur at point c. Other known distances are also suitable.

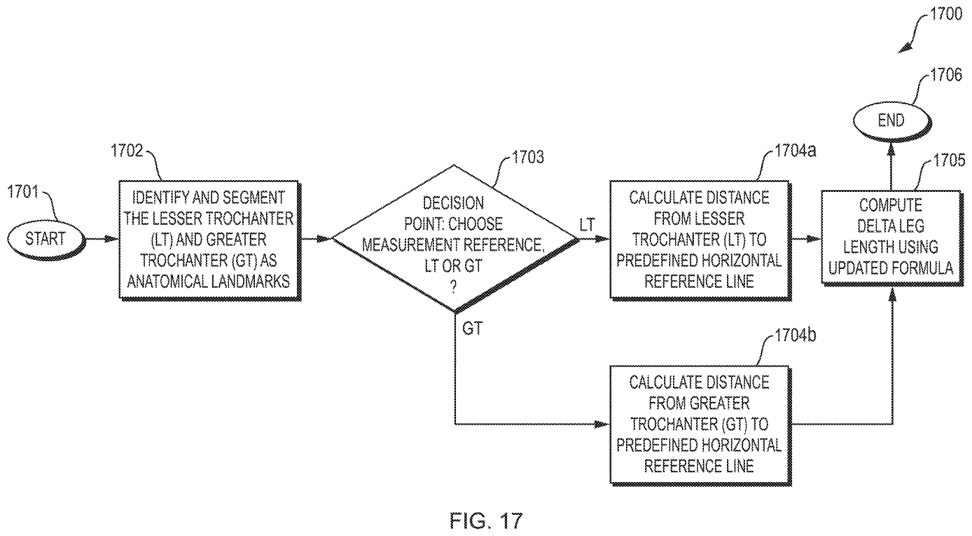

The process 1600 then ends at step 1606. According to an embodiment, a position of the newly defined 1605 horizontal line may be stored for subsequent use, such as with the process 1700 (described hereinbelow with respect to FIG. 17).

In an embodiment, the process 1600 may be employed as a secondary process in lieu of the process 500 for transferring a horizontal reference line to operative and non-operative AP hip X-rays (described hereinabove with respect to FIG. 5).

According to an embodiment, the process 1600 may autonomously apply a horizontal reference line, which may be defined as a line perpendicular (90°) to a long axis of a non-operative femur. In an embodiment, the line may be positioned at an equal distance along the Y-axis between an upper border of a GT and a lower border of an LT.

In an embodiment, if the LT is misidentified (e.g., at step 1603 (FIG. 16)), the process 1600 may instead place a horizontal reference line 2 cm below a tip of the non-operative GT along the Y-axis; other known distances are also suitable. According to an embodiment, the newly established reference line may be used to calculate “delta leg length” (DLL) at one or more of the three positions specified as part of the process 1700.

Provided below is example pseudocode in a Python-like language for determining alignments and/or orientations, applying rotations, extracting and/or processing femoral segments, calculating long axes, and rotating and/or aligning operative femurs according to an embodiment, which may be used as part of implementing the process 1600; other known programming languages are also suitable.
// Step 1: Identify key anatomical landmarks
Identify upper_greater_trochanter of non-operative femur
Identify lower_lesser_trochanter of non-operative femur
// Step 2: Compute the mid-point along the Y-axis
mid_point_Y=(upper_greater_trochanter_Y+lower_lesser_trochanter_Y)/2
// Step 3: Determine the long axis of the non-operative femur long_axis_femur=ComputeLongAxis(non-operative femur)
// Step 4: Draw a horizontal reference line reference_line=DrawPerpendicularLine(long_axis_femur, mid_point_Y)
// Step 5: Store the new reference line for further calculations StoreReferenceLine(reference_line, DLL_variables)//e.g., step 1704a or 1704b (FIG. 17)
// Step 6: Utilize this reference_line for DLL calculations ComputeDeltaLegLength(reference_line, DLL positions)

### Example Leg Length Difference Calculation

FIG. 17 is a flow diagram of an example process 1700 of calculating leg length discrepancy (e.g., DLL) according to an embodiment. In an embodiment, the process 1700 may be implemented by the system 110 (FIG. 1), such as in offset/length calculation module 160 (FIG. 1).

The process 1700 starts at step 1701 and identifies and/or segments a LT and/or GT as anatomical landmark(s) at step 1702.

At step 1703, the process 1700 selects the identified 1702 LT or GT as a measurement reference. It should be noted that, in an embodiment, the process 1700 may calculate and/or display/provide/report DLL using LT- and/or GT-based techniques. For example, if segmentation is unsuccessful for either LT or GT, then a value such as “N/A” will be reported for that technique and a calculation result will be reported for the other technique. If segmentation is successful for both LT and GT, then both calculation results will be reported.

For the LT at step 1703, the process 1700 calculates a distance from the LT to a predefined horizontal reference line at step 1704a. In an embodiment, step 1704a may include calculating or measuring a distance between the horizontal line and the lowest point of the LT in operative femur and/or non-operative femur images.

For the GT at step 1703, the process 1700 calculates a distance from the GT to the predefined horizontal reference line at step 1704b. According to an embodiment, step 1704b may include calculating or measuring a distance between the horizontal line and the tip of the GT in the operative femur and/or non-operative femur images.

At step 1705, the process 1700 computes DLL using an updated formula. For the LT at step 1704a, the formula is updated to subtract leg length non-operative (LLNOP) from leg length operative (LLOP). For the GT at step 1704b, the formula is updated to subtract LLOP from LLNOP.

The process 1700 then ends at step 1706. According to an embodiment, the process 1700 may optionally perform scaling and/or measurement conversion to ensure consistency across X-ray images (e.g., all images). For instance, the process 1700 may convert pixel distances into millimeters (mm) using an acetabular cup scaling factor, e.g., as determined by the process 900 (FIG. 9) or 1100 (FIG. 11).

An example embodiment may autonomously calculate separate leg lengths (e.g., three separate leg lengths) for an operative femur and/or non-operative femur using the x-axis or an alternative horizontal reference line, e.g., a line defined by the process 1600 (described hereinabove with respect to FIG. 16). One such embodiment may recognize landmark(s) of a tip of GT, upper LT, and/or and lower LT. In an embodiment, a delta of leg length operative (LLOP) to non-operative (LLNOP) may be determined for one or more of the preceding three landmarks (i.e., tip of GT, upper LT, and lower LT) and/or reported to a user.

Provided below is example pseudocode in a Python-like language for calculating leg length discrepancies, which may be used as part of implementing the process 1700; other known programming languages are also suitable.

```
# Calculate Leg Length Discrepancy
def calculate_leg_length (case_id, method=“LT”) ;
  overlay = Overlay.objects.get (case=case_id)
  if method = = “LT”:
    delta_leg_length = overlay.operative_lt_mm -
  overlay.non_operative_lt_mm
  else: # GT
    delta_leg_length = overlay.non_operative_gt_mm -
  overlay.operative_gt_mm
return delta_leg_length
```

### Example Femoral Offset Calculation

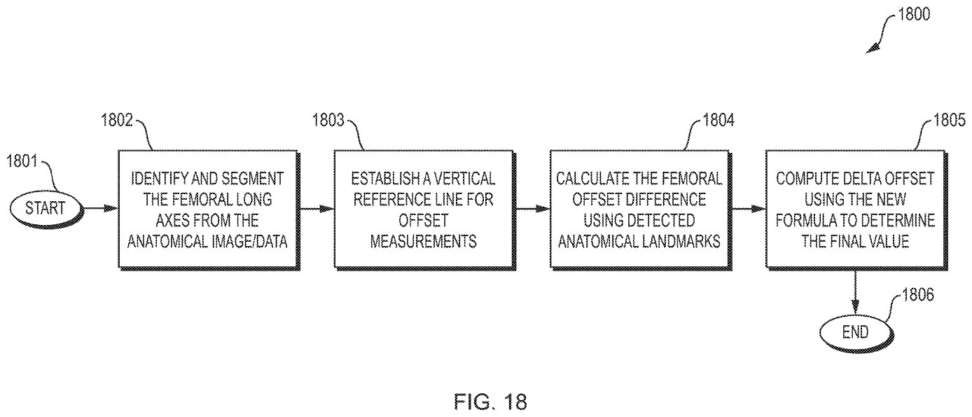

FIG. 18 is a flow diagram of an example process 1800 of femoral offset calculation according to an embodiment. In an embodiment, the process 1800 may be implemented by the system 110 (FIG. 1), such as in offset/length calculation module 160 (FIG. 1).

The process 1800 starts at step 1801 and identifies and/or segments femoral long axis(es) based on anatomical image(s)/data at step 1802.

At step 1803, the process 1800 determines or establishes a vertical reference line for offset measurements. In an embodiment, step 1803 may include identifying long axis(es) of operative and/or non-operative femurs and/or defining the vertical reference line (e.g., a singular vertical reference line) for accurate offset measurement.

Next, at step 1804, the process 1800 calculates a femoral offset difference from the determined 1803 vertical reference line using the detected 1802 anatomical landmark(s). According to an embodiment, step 1804 may include computing offset operative (OFOP) and/or offset non-operative (OFNOP) values.

At step 1805, the process 1800 computes a delta offset (DOF) using a new formula to determine a final value for the femoral offset. In an embodiment, step 1805 may use the following example formula: DOF=OFOP−OFNOP.

The process 1800 then ends at step 1806. According to an embodiment, the process 1800 may optionally perform scaling and/or measurement conversion. For instance, the process 1800 may convert pixel distances into mm using an acetabular cup scaling factor, e.g., as determined by the process 900 (FIG. 9) or 1100 (FIG. 11). In another embodiment, the process 1800 may display the computed 1805 DOF in a report (e.g., a final report).

An example embodiment may calculate DOF by determining an intersection of long axes of femurs (e.g., both femurs) and their difference(s) relative to one another along a horizontal reference line or alternative horizontal reference line. This technique may ensure consistency in femoral offset measurements.

Provided below is example pseudocode in a Python-like language for calculating femoral offsets, which may be used as part of implementing the process 1800; other known programming languages are also suitable.

```
# Calculate Femoral Offset
def calculate_femoral_offset (case_id) :
  overlay = Overlay.objects.get (case=case_id)
  delta_offset = overlay.operative_femoral_offset_mm -
overlay.non_operative_femoral_offset_mm
  return delta_offset
```

### Example Method Embodiment

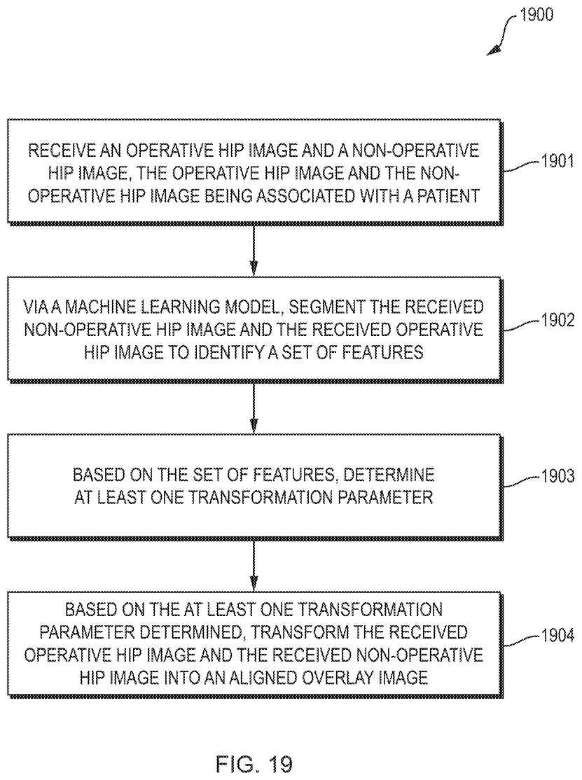

FIG. 19 is a flowchart of a method 1900 for ML-based image transformation. The method 1900 is computer-implemented and may be implemented using any computing device, e.g., a processor, or combination of computing devices known to those of skill in the art.

The method 1900 begins at step 1901 by receiving an operative hip image (e.g., 1000a (FIG. 10A), 1200a (FIG. 12A), or 1432 (FIGS. 14A & 14B)) and a non-operative hip image (e.g., 800a (FIG. 8A) or 1434 (FIGS. 14A & 14B)). The operative hip image and the non-operative hip image are associated with a patient. At step 1902, the method 1900 then, via an ML model (e.g., 120 (FIG. 1)), segments the received non-operative hip image and the received operative hip image to identify a set of features (e.g., 406a-406f (FIG. 4B)). Based on the set of features, at least one transformation parameter is determined at step 1903. In turn, at step 1904, based on the at least one transformation parameter determined, the method 1900 transforms the received operative hip image and the received non-operative hip image into an aligned overlay image (e.g., 1400b (FIG. 14B)).

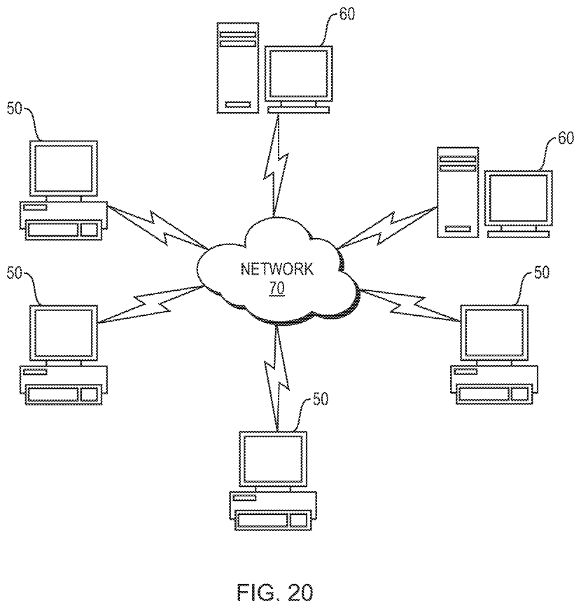

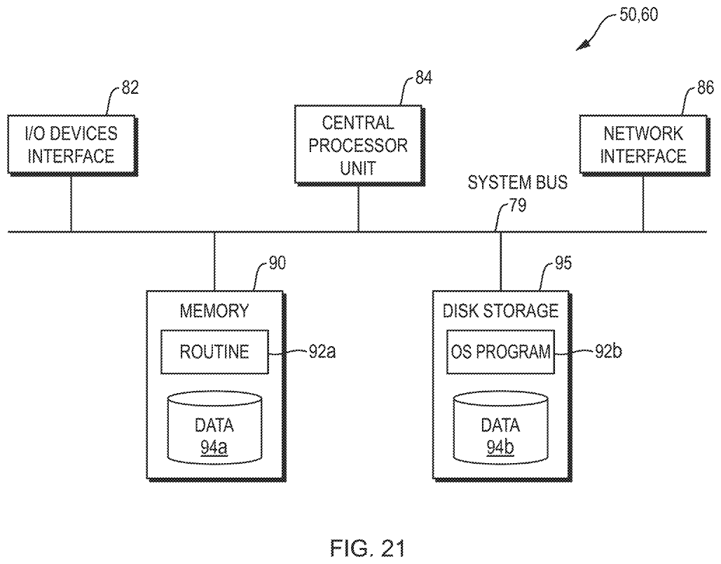

The method 1900 is computer-implemented and, as such, the functionality and effective operations, e.g., the receiving (1901), segmenting (1902), determining (1903), and transforming (1904), are automatically implemented by one or more digital processors. The method 1900 can also be implemented using any computer device or combination of computing devices known in the art. Among other examples, the method 1900 can be implemented using computer(s)/device(s) 50 and/or 60 described hereinbelow in relation to FIGS. 20 and 21.

In an example embodiment of the method 1900, transforming 1904 the received operative hip image and the received non-operative hip image may include inverting the received non-operative hip image. According to another example embodiment, transforming 1904 the received operative hip image and the received non-operative hip image may include overlaying the received operative hip image and the received non-operative hip image.

According to an embodiment of the method 1900, the at least one transformation parameter determined at step 1903 may include a horizontal translation parameter and a vertical translation parameter. Transforming 1904 the received operative hip image and the received non-operative hip image may include, based on the horizontal translation parameter and the vertical translation parameter, aligning the received operative hip image with the received non-operative hip image. In one such embodiment, the method 1900 may further include receiving a pelvis image. The pelvis image may be associated with the patient. The method 1900 may further include, via the ML model, segmenting the received pelvis image to identify a pubis feature. The method 1900 may further include, based on the pubis feature, determining a pubic orientation parameter. The method 1900 may further include, based on a predefined orientation parameter and the determined pubic orientation parameter, determining an intersection parameter. The method 1900 may further include, based on the pubis feature and the determined intersection parameter, determining the horizontal translation parameter and the vertical translation parameter. According to another such embodiment, the method 1900 may further include receiving a translation adjustment value from a user and, based on the received translation adjustment value, updating at least one of the determined horizontal translation parameter and vertical translation parameter.

In an example embodiment of the method 1900, the set of features may include at least one non-operative femoral feature and at least one operative femoral feature. The at least one transformation parameter determined at step 1903 may include a rotation parameter. Transforming 1904 the received operative hip image and the received non-operative hip image may include, based on a rotation center parameter and the rotation parameter, aligning the at least one operative femoral feature with the at least one non-operative femoral feature. According to one such embodiment, the at least one operative femoral feature may include at least one isolated femoral anatomic segment and at least one isolated femoral implant segment. Aligning the at least one operative femoral feature with the at least one non-operative femoral feature may include exclusively applying the rotation parameter to the at least one isolated femoral anatomic segment and at least one isolated femoral implant segment. In another such embodiment, the set of features further may include an acetabular cup feature. The method 1900 may further include, based on the acetabular cup feature, determining the rotation center parameter. According to yet another such embodiment, the method 1900 may further include receiving an acetabular cup size value and a distance value. The method 1900 may further include, based on the determined rotation center parameter, the received acetabular cup size value, and the received distance value, determining a scaled rotation center parameter. The method 1900 may further include updating the rotation center parameter based on the determined scaled rotation center parameter. In one such embodiment, determining the least one transformation parameter at step 1903 may include, based on a non-operative femoral axis value and an operative femoral axis value, determining the rotation parameter. The rotation parameter may be configured to align the non-operative femoral long axis parallel to the operative femoral long axis. According to another such embodiment, the method 1900 may further include receiving a rotation adjustment value from a user and, based on the received rotation adjustment value, updating the determined rotation parameter. In yet another such embodiment, the set of features may include a non-operative greater trochanter feature, a non-operative femur feature, an operative greater trochanter feature, and an operative femur feature. The method 1900 may further include, based on the non-operative greater trochanter feature and the non-operative femur feature, determining the non-operative femoral axis value. The method 1900 may further include, based on the operative greater trochanter feature and the operative femur feature, determining the operative femoral axis value.

According to an example embodiment of the method 1900, the set of features may include a non-operative upper greater trochanter feature, a non-operative lower lesser trochanter feature, an operative upper greater trochanter feature, and an operative lower lesser trochanter feature. The method 1900 may further include, based on a horizontal orientation parameter and (i) the non-operative upper greater trochanter feature and the operative upper greater trochanter feature or (ii) the non-operative lower lesser trochanter feature and the operative lower lesser trochanter feature, determining a leg length difference value. In one such embodiment, the method may further include receiving a pelvis image. The pelvis image may be associated with the patient. The method 1900 may further include, via the ML model, segmenting the received pelvis image to identify a teardrop feature and a pubis feature. The method 1900 may further include, based on the teardrop feature, determining the horizontal orientation parameter. The method 1900 may further include, based on the determined horizontal orientation parameter and the pubis feature, determining a vertical orientation parameter. According to another such embodiment, the method 1900 may further include, based on a non-operative femoral axis value, the non-operative upper greater trochanter feature, and the non-operative lower lesser trochanter feature, determining the horizontal orientation parameter.

In an example embodiment of the method 1900, the ML model may include at least one CNN. The at least one CNN may preferably include a YOLOv8 model.

According to an example embodiment, the method 1900 may further include outputting the aligned overlay image on a display.

In an example embodiment of the method 1900, receiving 1901 the operative hip image and the non-operative hip image may include obtaining hip X-ray data associated with the patient and processing the obtained hip X-ray data to generate the operative hip image and the non-operative hip image. According to one such embodiment, the obtained hip X-ray data may be interoperative data of the patient acquired from a C-arm based X-ray device. The method 1900 may further include displaying the obtained hip X-ray data on a user interface in real-time.

According to an example embodiment, receiving 1901 the operative hip image and the non-operative hip image may further include: (1) obtaining operative hip X-ray data associated with the patient; (2) processing the obtained operative hip X-ray data to generate the operative hip image; (3) obtaining non-operative hip X-ray data associated with the patient; and (4) processing the obtained non-operative hip X-ray data to generate the non-operative hip image. In one such embodiment, the obtained operative hip X-ray data and obtained non-operative hip X-ray data may be interoperative data of the patient acquired from a C-arm based X-ray device. The method 1900 may further include displaying the obtained operative hip X-ray data and obtained non-operative hip X-ray data on a user interface in real-time.

### Computer Support

FIG. 20 is a schematic view of a computer network in which embodiments may be implemented. Client computer(s)/devices 50 and server computer(s) 60 provide processing, storage, and input/output (I/O) devices executing application programs and the like. Client computer(s)/device(s) 50 can also be linked through communications network 70 to other computing devices, including other client device(s)/processor(s) 50 and server computer(s) 60. The communications network 70 can be part of a remote access network, a global network (e.g., the Internet), cloud computing servers or service, a worldwide collection of computers, local area or wide area networks, and gateways that currently use respective protocols (e.g., TCP/IP, Bluetooth®, etc.) to communicate with one another. Other electronic device/computer network architectures are also suitable.

FIG. 21 is a block diagram illustrating an example embodiment of a computer node (e.g., client processor(s)/device(s) 50 or server computer(s) 60) in the computer network 70 of FIG. 20. Each computer node 50, 60 contains system bus 79, where a bus is a set of hardware lines used for data transfer among components of a computer or processing system. The system bus 79 is essentially a shared conduit that connects different elements of a computer system (e.g., processor, disk storage, memory, I/O ports, network ports, etc.) that enables transfer of information between the elements. Attached to the system bus 79 is an I/O devices interface 82 for connecting various input and output devices (e.g., keyboard, mouse, display(s), printer(s), speaker(s), etc.) to the computer node 50, 60. A network interface 86 allows the computer node to connect to various other devices attached to a network (e.g., the network 70 of FIG. 20). A memory 90 provides volatile storage for computer software instructions 92a and data 94a used to implement embodiments of the present disclosure (e.g., the architecture 100 of FIG. 1, the process 200 of FIG. 2, the process 300 of FIG. 3, the process 500 of FIG. 5, the process 700 of FIG. 7, the process 900 of FIG. 9, the process 1100 of FIG. 11, the process 1300 of FIG. 13, the process 1500 of FIG. 15, the process 1600 of FIG. 16, the process 1700 of FIG. 17, the process 1800 of FIG. 18, the method 1900 of FIG. 19, etc.). A disk storage 95 provides non-volatile storage for the computer software instructions 92b and data 94b used to implement an embodiment of the present disclosure. A central processor unit 84 is also attached to the system bus 79 and provides for execution of computer instructions.

In an embodiment, the processor routines 92a-92b and data 94a-94b are a computer program product (generally referenced as 92), including a non-transitory, computer readable medium (e.g., a removable storage medium such as DVD-ROM(s), CD-ROM(s), diskette(s), tape(s), etc.) that provides at least a portion of the software instructions for the disclosure system. The computer program product 92 can be installed by any suitable software installation procedure, as is well known in the art. In another embodiment, at least a portion of the software instructions may also be downloaded over a cable, communication, and/or wireless connection. In other embodiments, the disclosure programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present disclosure routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other networks (such as the network 70 of FIG. 20). In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of the computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium, and the like.

In other embodiments, the program product 92 may be implemented as a so-called Software as a Service (SaaS), or other installation or communication supporting end-users.

Embodiments or aspects thereof may be implemented in the form of hardware including but not limited to hardware circuitry, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A computer-implemented method for machine learning (ML)-based image transformation, the computer-implemented method comprising:
receiving an operative hip image and a non-operative hip image, the operative hip image and the non-operative hip image being associated with a patient;
via an ML model, segmenting the received non-operative hip image and the received operative hip image to identify a set of features;
based on the set of features, determining at least one transformation parameter; and
based on the at least one transformation parameter determined, transforming the received operative hip image and the received non-operative hip image into an aligned overlay image;
wherein the set of features includes at least one non-operative femoral feature and at least one operative femoral feature, wherein the at least one transformation parameter determined includes a rotation parameter, and wherein transforming the received operative hip image and the received non-operative hip image includes:
based on a rotation center parameter and the rotation parameter, aligning the at least one operative femoral feature with the at least one non-operative femoral feature;
wherein the set of features further includes an acetabular cup feature, and further comprising:
based on the acetabular cup feature, determining the rotation center parameter.

2. The computer-implemented method of claim 1, wherein transforming the received operative hip image and the received non-operative hip image includes:
inverting the received non-operative hip image.

3. The computer-implemented method of claim 1, wherein transforming the received operative hip image and the received non-operative hip image includes:
overlaying the received operative hip image and the received non-operative hip image.

4. The computer-implemented method of claim 1, wherein the at least one transformation parameter determined includes a horizontal translation parameter and a vertical translation parameter, and wherein transforming the received operative hip image and the received non-operative hip image includes:
based on the horizontal translation parameter and the vertical translation parameter, aligning the received operative hip image with the received non-operative hip image.

5. The computer-implemented method of claim 4, further comprising:
receiving a pelvis image, the pelvis image being associated with the patient;
via the ML model, segmenting the received pelvis image to identify a pubis feature;
based on the pubis feature, determining a pubic orientation parameter;
based on a predefined orientation parameter and the determined pubic orientation parameter, determining an intersection parameter; and
based on the pubis feature and the determined intersection parameter, determining the horizontal translation parameter and the vertical translation parameter.

6. The computer-implemented method of claim 5, further comprising:
receiving a translation adjustment value from a user; and
based on the received translation adjustment value, updating at least one of the determined horizontal translation parameter and vertical translation parameter.

7. (canceled)

8. The computer-implemented method of claim 1, wherein the at least one operative femoral feature includes at least one isolated femoral anatomic segment and at least one isolated femoral implant segment, and wherein aligning the at least one operative femoral feature with the at least one non-operative femoral feature includes:
exclusively applying the rotation parameter to the at least one isolated femoral anatomic segment and at least one isolated femoral implant segment.

9. (canceled)

10. The computer-implemented method of claim 1, further comprising:
receiving an acetabular cup size value and a distance value;
based on the determined rotation center parameter, the received acetabular cup size value, and the received distance value, determining a scaled rotation center parameter; and
updating the rotation center parameter based on the determined scaled rotation center parameter.

11. The computer-implemented method of claim 1, wherein determining the at least one transformation parameter includes:
based on a non-operative femoral axis value and an operative femoral axis value, determining the rotation parameter, the rotation parameter being configured to align the non-operative femoral long axis parallel to the operative femoral long axis.

12. The computer-implemented method of claim 11, further comprising:
receiving a rotation adjustment value from a user; and
based on the received rotation adjustment value, updating the determined rotation parameter.

13. The computer-implemented method of claim 11, wherein the set of features includes a non-operative greater trochanter feature, a non-operative femur feature, an operative greater trochanter feature, and an operative femur feature, and further comprising:
based on the non-operative greater trochanter feature and the non-operative femur feature, determining the non-operative femoral axis value; and
based on the operative greater trochanter feature and the operative femur feature, determining the operative femoral axis value.

14. The computer-implemented method of claim 1, wherein the set of features includes a non-operative upper greater trochanter feature, a non-operative lower lesser trochanter feature, an operative upper greater trochanter feature, and an operative lower lesser trochanter feature, and further comprising:
based on a horizontal orientation parameter and (i) the non-operative upper greater trochanter feature and the operative upper greater trochanter feature or (ii) the non-operative lower lesser trochanter feature and the operative lower lesser trochanter feature, determining a leg length difference value.

15. The computer-implemented method of claim 14, further comprising:
receiving a pelvis image, the pelvis image being associated with the patient;
via the ML model, segmenting the received pelvis image to identify a teardrop feature and a pubis feature;
based on the teardrop feature, determining the horizontal orientation parameter; and
based on the determined horizontal orientation parameter and the pubis feature, determining a vertical orientation parameter.

16. The computer-implemented method of claim 14, further comprising:
based on a non-operative femoral axis value, the non-operative upper greater trochanter feature, and the non-operative lower lesser trochanter feature, determining the horizontal orientation parameter.

17. The computer-implemented method of claim 1, wherein the ML model includes at least one convolutional neural network (CNN), and preferably wherein the at least one CNN includes a YOLOv8 model.

18. The computer-implemented method of claim 1, further comprising:
outputting the aligned overlay image on a display.

19. The computer-implemented method of claim 1, wherein receiving the operative hip image and the non-operative hip image includes:
obtaining hip X-ray data associated with the patient; and
processing the obtained hip X-ray data to generate the operative hip image and the non-operative hip image.

20. The computer-implemented method of claim 19, wherein the obtained hip X-ray data is interoperative data of the patient acquired from a C-arm based X-ray device, and further comprising:
displaying the obtained hip X-ray data on a user interface in real-time.

21. The computer-implemented method of claim 1, wherein receiving the operative hip image and the non-operative hip image includes:
obtaining operative hip X-ray data associated with the patient;
processing the obtained operative hip X-ray data to generate the operative hip image;
obtaining non-operative hip X-ray data associated with the patient; and
processing the obtained non-operative hip X-ray data to generate the non-operative hip image.

22. The computer-implemented method of claim 21, wherein the obtained operative hip X-ray data and obtained non-operative hip X-ray data are interoperative data of the patient acquired from a C-arm based X-ray device, and further comprising:
displaying the obtained operative hip X-ray data and obtained non-operative hip X-ray data on a user interface in real-time.

23. The computer-implemented method of claim 1, wherein the at least one transformation parameter determined includes a horizontal translation parameter and a vertical translation parameter, and further comprising:
based on the acetabular cup feature, the horizontal translation parameter, and the vertical translation parameter, determining an acetabular cup inclination parameter; and
based on the acetabular cup feature, determining an acetabular cup anteversion parameter.

24. The computer-implemented method of claim 1, further comprising:
receiving a pelvis image, the pelvis image being associated with the patient;
wherein the segmenting includes, via the ML model, segmenting the received non-operative hip image, the received operative hip image, and the received pelvis image to identify the set of features.

25. A computer-based system for machine learning (ML)-based image transformation, the computer-based system comprising:
a processor; and
a memory with computer code instructions stored thereon, the processor and the memory, with the computer code instructions, being configured to cause the computer-based system to:
receive an operative hip image and a non-operative hip image, the operative hip image and the non-operative hip image being associated with a patient;
via an ML model, segment the received non-operative hip image and the received operative hip image to identify a set of features;
based on the set of features, determine at least one transformation parameter; and
based on the at least one transformation parameter determined, transform the received operative hip image and the received non-operative hip image into an aligned overlay image;
wherein the set of features includes at least one non-operative femoral feature and at least one operative femoral feature, wherein the at least one transformation parameter determined includes a rotation parameter, and wherein transforming the received operative hip image and the received non-operative hip image includes:
based on a rotation center parameter and the rotation parameter, aligning the at least one operative femoral feature with the at least one non-operative femoral feature;
wherein the set of features further includes an acetabular cup feature, and wherein the processor and the memory, with the computer code instructions, are further configured to cause the computer-based system to:
based on the acetabular cup feature, determine the rotation center parameter.

26. A computer program product for machine learning (ML)-based image transformation, the computer program product comprising a non-transitory computer-readable medium with computer code instructions stored thereon, the computer code instructions being configured, when executed by a processor, to cause an apparatus associated with the processor to:
receive an operative hip image and a non-operative hip image, the operative hip image and the non-operative hip image being associated with a patient;
via an ML model, segment the received non-operative hip image and the received operative hip image to identify a set of features;
based on the set of features, determine at least one transformation parameter; and
based on the at least one transformation parameter determined, transform the received operative hip image and the received non-operative hip image into an aligned overlay image;
wherein the set of features includes at least one non-operative femoral feature and at least one operative femoral feature, wherein the at least one transformation parameter determined includes a rotation parameter, and wherein transforming the received operative hip image and the received non-operative hip image includes:
based on a rotation center parameter and the rotation parameter, aligning the at least one operative femoral feature with the at least one non-operative femoral feature;
wherein the set of features further includes an acetabular cup feature, and wherein the computer code instructions are further configured, when executed by a processor, to cause an apparatus associated with the processor to:
based on the acetabular cup feature, determine the rotation center parameter.

27. A computer-implemented method for machine learning (ML)-based image transformation, the computer-implemented method comprising:
receiving an operative hip image and a non-operative hip image, the operative hip image and the non-operative hip image being associated with a patient;
via an ML model, segmenting the received non-operative hip image and the received operative hip image to identify a set of features;
based on the set of features, determining at least one transformation parameter; and
based on the at least one transformation parameter determined, transforming the received operative hip image and the received non-operative hip image into an aligned overlay image;
wherein the set of features includes a non-operative upper greater trochanter feature, a non-operative lower lesser trochanter feature, an operative upper greater trochanter feature, and an operative lower lesser trochanter feature, and further comprising:
based on a horizontal orientation parameter and (i) the non-operative upper greater trochanter feature and the operative upper greater trochanter feature or (ii) the non-operative lower lesser trochanter feature and the operative lower lesser trochanter feature, determining a leg length difference value.

28. The computer-implemented method of claim 27, further comprising:
receiving a pelvis image, the pelvis image being associated with the patient;
via the ML model, segmenting the received pelvis image to identify a teardrop feature and a pubis feature;
based on the teardrop feature, determining the horizontal orientation parameter; and
based on the determined horizontal orientation parameter and the pubis feature, determining a vertical orientation parameter.

29. The computer-implemented method of claim 27, further comprising:
based on a non-operative femoral axis value, the non-operative upper greater trochanter feature, and the non-operative lower lesser trochanter feature, determining the horizontal orientation parameter.
